(19) United States
(12) Patent Application Publication
(10) Pub. No.: US 2021/0093336 A1
(43) Pub. Date: Apr. 1, 2021

(54) EMBOLIC RETRIEVAL CATHETER

(71) Applicant: Incept, LLC, Campbell, CA (US)

(72) Inventors: Chad C. Roue, San Jose, CA (US); Yi Yang, San Francisco, CA (US); Farhad Khosravi, Los Altos, CA (US)

(21) Appl. No.: 17/036,258

(22) Filed: Sep. 29, 2020

Related U.S. Application Data
(63) Continuation-in-part of application No. 16/589,563, filed on Oct. 1, 2019.
(60) Provisional application No. 63/026,898, filed on May 19, 2020.

(51) Int. Cl. A61B 17/221

(52) U.S. Cl. CPC A61B 17/221; A61B 2017/00867; A61B 2017/2215

(57) ABSTRACT

Disclosed are systems for removing embolic material from an intravascular site. An elongate, flexible tubular body has a tubular side wall defining at least one axially extending lumen. An axial restraint is carried by an inner surface of the side wall and exposed to the lumen. A rotatable core wire is extendable through the lumen, and may include an obstruction engaging tip. The core wire has a limit with a distally facing bearing surface for rotatably engaging the restraint. The limit and the restraint are configured to permit rotation of the core wire but limit distal advance of the tip to a predetermined relationship with the distal end of the tubular body.

### CROSS-REFERENCE TO RELATED APPLICATIONS

This application is a continuation-in-part of U.S. patent application Ser. No. 16/589,563, filed Oct. 1, 2019, and also claims the benefit of priority under 35 U.S.C. § 119(e) of U.S. Provisional Application No. 63/026,898, filed May 19, 2020, the entirety of each of which is hereby incorporated by reference herein. Additionally, International Patent Application No. PCT/US2019/029709, filed on Apr. 29, 2019, is hereby incorporated by reference herein.

### BACKGROUND

Stroke is the third most common cause of death in the United States and the most disabling neurologic disorder. Approximately 700,000 patients suffer from stroke annually. Stroke is a syndrome characterized by the acute onset of a neurological deficit that persists for at least 24 hours, reflecting focal involvement of the central nervous system, and is the result of a disturbance of the cerebral circulation. Its incidence increases with age. Risk factors for stroke include systolic or diastolic hypertension, hypercholesterolemia, cigarette smoking, heavy alcohol consumption, and oral contraceptive use.

Hemorrhagic stroke accounts for 20% of the annual stroke population. Hemorrhagic stroke often occurs due to rupture of an aneurysm or arteriovenous malformation bleeding into the brain tissue, resulting in cerebral infarction. The remaining 80% of the stroke population are ischemic strokes and are caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. When a patient presents with neurological symptoms and signs which resolve completely within 1 hour, the term transient ischemic attack (TIA) is used. Etiologically, TIA and stroke share the same pathophysiologic mechanisms and thus represent a continuum based on persistence of symptoms and extent of ischemic insult.

Emboli occasionally form around the valves of the heart or in the left atrial appendage during periods of irregular heart rhythm and then are dislodged and follow the blood flow into the distal regions of the body. Those emboli can pass to the brain and cause an embolic stroke. As will be discussed below, many such occlusions occur in the middle cerebral artery (MCA), although such is not the only site where emboli come to rest.

When a patient presents with neurological deficit, a diagnostic hypothesis for the cause of stroke can be generated based on the patient's history, a review of stroke risk factors, and a neurologic examination. If an ischemic event is suspected, a clinician can tentatively assess whether the patient has a cardiogenic source of emboli, large artery extracranial or intracranial disease, small artery intraparenchymal disease, or a hematologic or other systemic disorder. A head CT scan is often performed to determine whether the patient has suffered an ischemic or hemorrhagic insult. Blood would be present on the CT scan in subarachnoid hemorrhage, intraparenchymal hematoma, or intraventricular hemorrhage.

Traditionally, emergent management of acute ischemic stroke consisted mainly of general supportive care, e.g. hydration, monitoring neurological status, blood pressure control, and/or anti-platelet or anti-coagulation therapy. In 1996, the Food and Drug Administration approved the use of Genentech Inc.'s thrombolytic drug, tissue plasminogen activator (t-PA) or Activase®, for treating acute stroke. A randomized, double-blind trial, the National Institute of Neurological Disorders and t-PA Stroke Study, revealed a statistically significant improvement in stoke scale scores at 24 hours in the group of patients receiving intravenous t-PA within 3 hours of the onset of an ischemic stroke. Since the approval of t-PA, an emergency room physician could, for the first time, offer a stroke patient an effective treatment besides supportive care.

However, treatment with systemic t-PA is associated with increased risk of intracerebral hemorrhage and other hemorrhagic complications. Patients treated with t-PA were more likely to sustain a symptomatic intracerebral hemorrhage during the first 36 hours of treatment. The frequency of symptomatic hemorrhage increases when t-PA is administered beyond 3 hours from the onset of a stroke. Besides the time constraint in using t-PA in acute ischemic stroke, other contraindications include the following: if the patient has had a previous stroke or serious head trauma in the preceding 3 months, if the patient has a systolic blood pressure above 185 mm Hg or diastolic blood pressure above 110 mmHg, if the patient requires aggressive treatment to reduce the blood pressure to the specified limits, if the patient is taking anticoagulants or has a propensity to hemorrhage, and/or if the patient has had a recent invasive surgical procedure. Therefore, only a small percentage of selected stroke patients are qualified to receive t-PA.

Obstructive emboli have also been mechanically removed from various sites in the vasculature for years. Mechanical therapies have involved capturing and removing the clot, dissolving the clot, disrupting and suctioning the clot, and/or creating a flow channel through the clot. One of the first mechanical devices developed for stroke treatment is the MERCI Retriever System (Concentric Medical, Redwood City, Calif.). A balloon-tipped guide catheter is used to access the internal carotid artery (ICA) from the femoral artery. A microcatheter is placed through the guide catheter and used to deliver the coil-tipped retriever across the clot and is then pulled back to deploy the retriever around the clot. The microcatheter and retriever are then pulled back, with the goal of pulling the clot, into the balloon guide catheter while the balloon is inflated and a syringe is connected to the balloon guide catheter to aspirate the guide catheter during clot retrieval. This device has had initially positive results as compared to thrombolytic therapy alone.

Other thrombectomy devices utilize expandable cages, baskets, or snares to capture and retrieve clot. Temporary stents, sometimes referred to as stentrievers or revascularization devices, are utilized to remove or retrieve clot as well as restore flow to the vessel. A series of devices using active laser or ultrasound energy to break up the clot have also been utilized. Other active energy devices have been used in conjunction with intra-arterial thrombolytic infusion to accelerate the dissolution of the thrombus. Many of these devices are used in conjunction with aspiration to aid in the removal of the clot and reduce the risk of emboli. Suctioning of the clot has also been used with single-lumen catheters and syringes or aspiration pumps, with or without adjunct disruption of the clot. Devices which apply powered fluid vortices in combination with suction have been utilized to improve the efficacy of this method of thrombectomy. Finally, balloons or stents have been used to create a patent lumen through the clot when clot removal or dissolution was not possible.

Notwithstanding the foregoing, there remains a need for new devices and methods for treating vasculature occlusions in the body, including acute ischemic stroke and occlusive cerebrovascular disease.

### SUMMARY

There is provided in accordance with one aspect of the invention, a system for removing embolic material from an intravascular site. The system comprises an elongate, flexible tubular body, having a proximal end, a distal end, and a tubular side wall defining at least one lumen extending axially there through. An axial restraint is carried by the side wall and exposed to the lumen. A rotatable core wire is extendable through the lumen, the core wire having a proximal end and a distal end. A limit is carried by the core wire, the limit having a bearing surface for rotatably engaging the restraint, and a clot grabbing tip is provided on the distal end of the core wire. The limit and the restraint are configured to permit rotation of the core wire but limit distal advance of the tip to no more than about 6 mm beyond the distal end of the tubular body.

The limit and the restraint may be configured to permit rotation of the core wire but limit distal advance of the tip to no more than about 3 mm beyond the distal end of the tubular body. The clot grabbing tip may comprises a helical thread. The limit and the restraint may be configured to permit rotation of the core wire but limit distal advance of the tip to expose between about one and three full revolutions of the thread beyond the distal end of the tubular body.

The axial restraint may comprise a proximally facing bearing surface. The axial restraint may comprise a radially inwardly extending projection. The axial restraint may comprise an annular flange. The limit may comprise a distally facing bearing surface. The limit may comprise a radially outwardly extending projection. The radially outwardly extending projection may be configured for sliding contact with the restraint.

A proximal bearing surface on the axial restraint may be within about 30 cm from the distal end of the tubular body. The proximal bearing surface may be within the range of from about 4 cm to about 12 cm from the distal end of the tubular body. The limit may be positioned within about the distal most 25% of the core wire length.

The helical thread may have a greatest major diameter that is no more than about 90% of the inside diameter of the lumen, leaving an annular flow path between the tip and the inner surface of the side wall. The helical thread may have a blunt outer edge.

The core wire may be removably positionable within the tubular body. The system may further comprise a handle configured for manual rotation of the core wire.

The helical thread may extend through no more than about eight full revolutions. The helical thread may have a major diameter that increases in a proximal direction from a first diameter near the distal tip to a second, greatest major diameter, and then decreases proximally of the greatest major diameter to a third diameter. An inside diameter of the tubular body adjacent the clot grabbing tip may be at least about 0.015″ greater than a maximum outside diameter of the tip.

There is provided in accordance with one aspect of the invention, a torque transmission system for rotationally orienting a distal end of a catheter. The system comprises an elongate, flexible tubular body, having a proximal end, a distal end, and a tubular side wall defining at least one lumen extending axially there through. A first engagement surface is carried by the side wall and exposed to the lumen. A torque wire is extendable through the lumen, the torque wire having a proximal end and a distal end. A second engagement surface is carried by the torque wire. Distal advance of the torque wire brings the second engagement surface into rotational coupling engagement with the first engagement surface such that rotation of the torque wire in at least a first direction causes rotation of the distal end of the catheter.

The first engagement surface may comprise at least one inclined surface, and may be carried by a radially inwardly extending projection. The projection may comprise a ring positioned in the lumen. The second engagement surface may comprise a distally facing surface, which may be inclined relative to a longitudinal axis of the catheter.

In accordance with another aspect of the invention, there is provided a system for removing embolic material from an intravascular site. The system comprises an elongate, flexible tubular body, having a proximal end, a distal end, and a tubular side wall defining at least one lumen extending axially there through. A first engagement surface is carried by the side wall and exposed to the lumen. A tap wire is extendable through the lumen, the tap wire having a proximal end and a distal end. A second engagement surface is carried by the tap wire. Distal advance of the tap wire brings the second engagement surface into contact with the first engagement surface and transfers momentum from the tap wire to the distal end of the tubular body.

The first engagement surface may comprise a proximally facing surface, and may be carried by a radially inwardly extending projection. The first engagement surface may comprise an annular flange. The second engagement surface may comprise a distally facing surface, which may be carried by a distal end of the tap wire. The distally facing surface may be on a hammer head carried by the wire.

In accordance with a further aspect of the present invention, there is provided a torque transmission system for rotationally orienting a distal end of a catheter. The system comprises an elongate, flexible tubular body, having a proximal end, a distal end, and a tubular side wall defining at least one lumen extending axially there through. A first connector is provided on the side wall and exposed to the lumen. A torque wire is extendable through the lumen, the torque wire having a proximal end and a distal end. A second, complementary connector is carried by the torque wire. Coupling the first and second connectors enables rotation of the distal end of the catheter in response to rotation of the torque wire.

The first connector may comprises at least one angled tooth, or a radially inwardly extending projection. The projection may comprise a ring positioned in the lumen. The ring may comprise at least two angled teeth extending in a proximal direction. The second connector may comprise a distally facing surface carried by the torque wire. The distally facing surface may comprise at least one inclined surface. The second connector may be radially outwardly movable, and may comprise an inflatable balloon and the first connector may comprise an inner surface on the side wall. The first connector may comprise the side wall of an axially extending slot configured to receive a projection on the torque wire.

There is also provided a method of rotationally orienting a catheter. The method comprises the steps of advancing a catheter to a site in a body lumen, the catheter having a central lumen and a distal end. A torque wire is advanced into the lumen. A first connector on the torque wire is engaged with a second connector on the catheter, and rotating the torque wire causes a rotation of the distal end of the catheter.

Any feature, structure, or step disclosed herein can be replaced with or combined with any other feature, structure, or step disclosed herein, or omitted. Further, for purposes of summarizing the disclosure, certain aspects, advantages, and features of the embodiments have been described herein. It is to be understood that not necessarily any or all such advantages are achieved in accordance with any particular embodiment disclosed herein. No individual aspects of this disclosure are essential or indispensable. Further features and advantages of the embodiments will become apparent to those of skill in the art in view of the Detailed Description which follows when considered together with the attached drawings and claims.

### BRIEF DESCRIPTION OF THE DRAWINGS

FIG. 1A is a side elevational view of a catheter having an internal stop ring.

FIG. 1B is a longitudinal cross section through the catheter of FIG. 1A, and detail view of the stop ring.

FIG. 1C is a side elevational view of an agitator having a complementary limit for engaging the stop ring of FIGS. 1A and 1B.

FIG. 1D is a side elevational view of a distal portion of the agitator of FIG. 24C.

FIG. 1E is a longitudinal cross section through the agitator of FIG. 1D.

FIG. 1F is a perspective cut away view of a distal portion of the agitator of FIG. 24C.

FIG. 1G is a transverse cross section through a distal stopper carried by the agitator.

FIG. 1H is a transverse cross section through an alternative distal stopper.

FIG. 2A is a side elevational view of a torque wire for rotating the distal end of an aspiration catheter.

FIGS. 2B and 2C show torque wires in relation to distal stoppers configured as torque rings.

FIG. 3A is a side elevational view of an obstruction engaging wire that is axially unrestrained within an access catheter.

FIGS. 3B and 3C are side elevational views of an obstruction engaging wire with a limited distal range of motion beyond the distal end of the catheter.

FIG. 4 is a side elevational view of a driver wire for use with the catheter of FIG. 1A and 1B.

FIGS. 5A and 5B are side elevational views of a blood flow assisted wire for use with a catheter having a stop ring.

FIG. 5C shows a steerable wire for use with a catheter having a stop ring.

FIG. 5D is a detail view of the distal end of the steerable wire of FIG. 5C.

FIG. 5E is a side elevational view of the wire of FIG. 5C, in a deflected configuration.

FIGS. 6A-6B show various distal cap embodiments for coupling the core wire to an annular flange of the agitator tip.

FIG. 7A illustrates a cross-sectional elevational view of a catheter wall according to an embodiment.

FIG. 7B illustrates a cross-sectional elevational view of a catheter wall according to another embodiment, showing one or more axially extending filaments.

FIG. 7C describes a side elevational view of a catheter.

FIG. 7D illustrates a cross-sectional view through a catheter with a side wall having one or more axially extending filaments.

FIG. 7E is a side elevational cross section through an angled distal catheter or extension tube tip.

FIG. 8A depicts a side elevational view of a catheter according to one embodiment.

FIG. 8B describes a cross-sectional elevational view taken along the line A-A of FIG. 8A.

FIG. 8C illustrates a cross-sectional view taken along the line B-B of FIG. 8A.

FIG. 9A depicts a side elevational view of a catheter according to another embodiment.

FIG. 9B describes a cross-sectional elevational view taken along the line A-A of FIG. 9A, showing one or more axially extending filaments.

FIG. 9C illustrates a cross-sectional view taken along the line B-B of FIG. 9A, showing one or more axially extending filaments.

FIGS. 10A-10B are side elevational cross sections through a blunted, angled distal catheter or extension tube tip.

FIG. 11A is a side elevational cross section through a sinusoidal shaped distal catheter or extension tube tip.

FIG. 11B is a perspective view of a sinusoidal shaped distal catheter or extension tube tip.

FIG. 12 shows a schematic of a computational fluid dynamics (CFD) velocity field model.

FIG. 13A shows an image of a numerical simulation of clot aspiration using the model of FIG. 12, illustrating an initial state after some aspiration has occurred.

FIG. 13B shows an image of a numerical simulation of clot aspiration using the model of FIG. 12, illustrating active aspiration.

FIG. 14 shows a graphical representation of a percent increase in aspirated material for the catheter distal tip shapes shown in FIGS. 7E, and 10A-11B as a percent improvement over the flat or planar catheter distal tip shape shown in FIG. 3A.

FIG. 15A depicts a CFD velocity field profile for a flat or planar catheter distal tip.

FIG. 15B depicts a CFD velocity field profile for a catheter distal tip having an angle of 30°.

FIG. 15C depicts a CFD velocity field profile for a catheter distal tip having an angle of 60°.

FIG. 16A shows an ingestion length for an angled distal catheter tip.

FIG. 16B shows an ingestion length for a blunted, angled distal catheter tip.

FIG. 17 shows a graphical representation of a percent increase in aspirated material for the catheter distal tip shapes shown in FIGS. 7E and 10A-11B, as a percent improvement over the flat, perpendicular catheter distal tip shape shown in FIG. 3A.

FIG. 18A illustrates a side elevational view of a progressively enhanced flexibility catheter according to an embodiment.

FIG. 18B is a proximal end view of the enhanced flexibility catheter of FIG. 18A.

FIG. 19 illustrates back-up support of the catheter in accordance with the present invention.

FIG. 20 depicts a graph of modulus or durometer of the catheter along the length of the catheter, from the proximal end to the distal end.

FIG. 21 depicts a graph of flexure test profiles of catheters in accordance with the present invention compared with conventional catheters.

### DETAILED DESCRIPTION OF THE PREFERRED EMBODIMENT

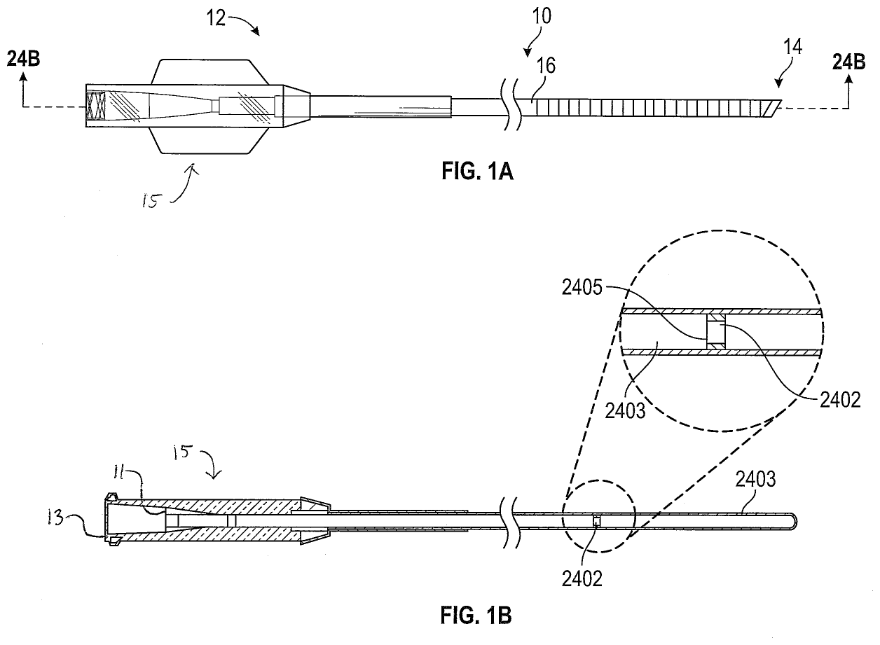

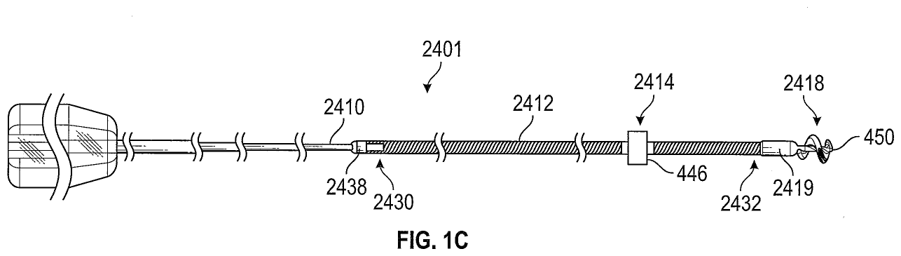

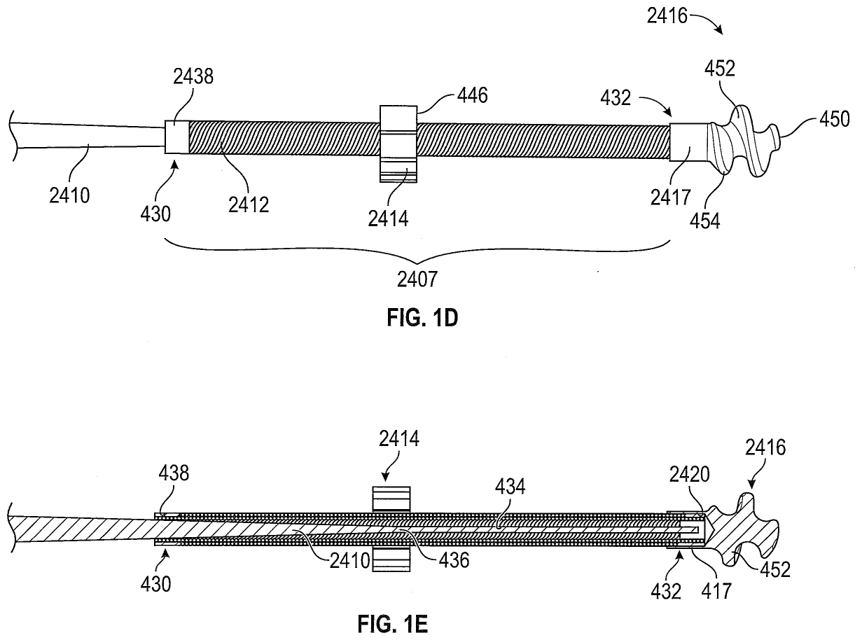

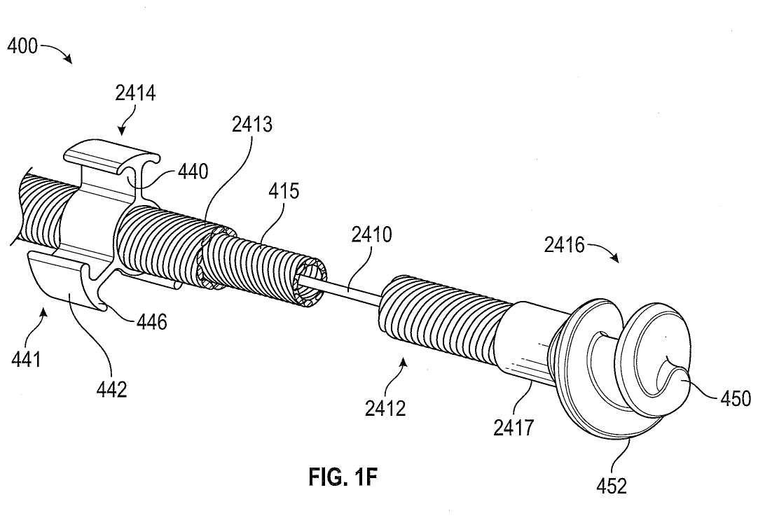

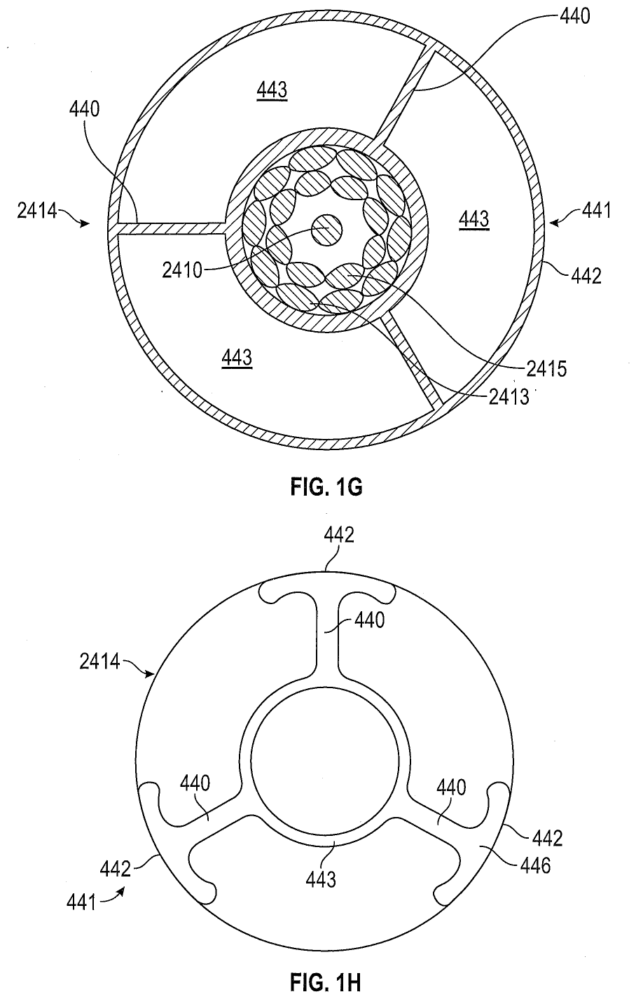

Referring to FIG. 1A, there is disclosed a catheter 10 in accordance with one aspect of the present invention. Although primarily described in the context of an elongate flexible catheter such as an embolic clot retrieval catheter with a single central lumen, catheters of the present invention can readily be modified to incorporate additional structures, such as permanent or removable column strength enhancing mandrels, two or more lumen such as to permit drug, contrast or irrigant infusion or to supply inflation media to an inflatable balloon carried by the catheter, or combinations of these features, as will be readily apparent to one of skill in the art in view of the disclosure herein. In addition, the present invention will be described primarily in the context of removing obstructive material from remote vasculature in the brain, but has applicability as an access catheter for delivery and removal of any of a variety of diagnostics or therapeutic devices with or without aspiration.

The catheters disclosed herein may readily be adapted for use throughout the body wherever it may be desirable to position a catheter configured to precisely limit distal advance of other internal catheter components or tools axially therethrough. For example, catheter shafts in accordance with the present invention may be dimensioned for use throughout either the venous or arterial side of the neuro vasculature, the coronary and peripheral vasculature, the gastrointestinal tract, the urethra, ureters, Fallopian tubes and other lumens and potential lumens, as well. Particular applications include clot removal in the cases of ischemic stroke, deep vein thrombosis and pulmonary embolism. The catheter structure of the present invention may also be used to provide minimally invasive percutaneous tissue access, such as for diagnostic or therapeutic access to a solid tissue target (e.g., breast or liver or brain diagnosis or therapy), delivery of laparoscopic tools or access to bones such as the spine for delivery of devices that benefit from precise axial control.

The catheter 10 generally comprises an elongate tubular body 16 extending between a proximal end 12 and a distal functional end 14. The length of the tubular body 16 depends upon the desired application. For example, lengths in the area of from about 120 cm to about 150 cm or more are typical for use in femoral access percutaneous transluminal coronary applications. Intracranial or other applications may call for a different catheter shaft length depending upon the vascular access site, as will be understood in the art.

Referring to FIG. 1B, any of the catheters disclosed herein may be provided with an axial restraint 2402 for cooperating with a complementary stopper on an interior component such as a rotational device to permit rotation of the device but limit the distal axial range of travel of the internal device. This allows precise positioning of the distal tip of the inner device with respect to the distal end of the catheter, decoupled from bending of the catheter shaft, and prevent the distal tip of the inner device from extending beyond a preset position such as the distal end of the catheter.

In the illustrated implementation, the distal restraint or restriction element 2402 comprises at least one projection extending radially inwardly from the inside surface of the tubular body, configured to restrict the inside diameter of the aspiration lumen and provide an interference bearing surface to engage a distal face carried by the agitator. The restraint may comprise one or two or three or four or more projections such as tabs, or, as illustrated, in FIG. 1B may comprise a continuous or split annular ring providing a substantially continuous annular proximally facing restraint surface. The proximal bearing surface 2405 of the axial restraint 2402 may be located within about 50 cm or 30 cm or 15 cm from the distal end of the tubular body, such as within the range of from about 4 cm to about 12 cm from the distal end.

Alternatively, the bearing surface 2405 may be provided at the proximal end of the catheter 10 depending upon desired performance and intended vasculature. For example, a proximal hub 15 may be provided with an interior bearing surface 11 to slidably engage a complementary distal bearing surface carried by the core wire. An external surface 13 carried by the hub 15, such as an annular surface surrounding the central lumen may alternatively be brought into sliding engagement with a complementary distal surface carried by the control wire handle, discussed below.

In order to optimize alignment of the distal rotatable tip of an inner device with the distal port of the catheter, and decouple that axial alignment from the tortuosity of the vascular path which otherwise changes the relative axial positions of the catheter exit port and the tip, the proximal bearing surface of the axial restraint is often within the range of from about 3 mm to about 50 mm, in some implementations about 5 mm to about 20 mm and in one implementation from about 6 mm to about 14 mm from the distal port on the catheter.

The distal restraint may be a metallic (e.g., nitinol, stainless steel, aluminum, etc.) or polymeric circular band or ring or protrusion 2402 mounted on or built into the interior surface of a sidewall of the catheter 2403 near the distal tip, the distal restriction element 2402 extending into the ID of the catheter. Further, the distal restriction element 2402 may be radiopaque for visibility under fluoroscopy. The distal restriction element 2402 carries a proximally facing surface 2405 for example an annular circumferential bearing surface that extends into the inner diameter of the catheter to interface with a distal stopper 2414 on the rotating assembly. See FIG. 1C. For example, the distal stopper 2414 may be a circular feature on the rotating assembly which interfaces with the distal restriction element 2402 of the catheter to stop the distal advancement and prevent distal tip displacement beyond the catheter distal tip.

In one implementation, in its relaxed form prior to securing within the catheter lumen, the ring 2402 is a C-shaped or cylinder shaped with an axially extending slit to form a split ring. The ring 2402 is compressed using a fixture that collapses the ring to a closed circle shape, allowing it to slide inside the (e.g., 0.071″) catheter. When the ring is released from the fixture, the ring expands radially to the largest diameter permitted by the inside diameter of the catheter. The radial force of the ring engages the insider surface of the catheter and resists axial displacement under the intended use applied forces. In another implementation, the ring is a fully closed, continuous annular structure (like a typical marker band) and its distal end is slightly flared in a radially outwardly direction to create a locking edge. The ring is inserted into the catheter from the distal end. The flared section with the locking edge keeps the ring in place when axial force is applied from the proximal side.

Alternatively, the distal restraint may be formed by forming a radially inwardly directed crimp into the catheter body, or introducing a bearing surface through the wall of the catheter and into communication with the central lumen such as by introducing at least one or two or more projections through the wall and into the lumen. The restraint may take the form of a concentric inner tube having an axial length of at least about 1 mm or 2 mm or more but generally less than about 5 cm or 3 cm or 2 cm. Depending upon the nature of the distal restraint, it may be secured by mechanical interference fit, friction fit, or a bond formed by any of a variety of bonding techniques including adhesive bonding and thermal bonding.

Referring to FIG. 1C and 1D, the inner member is in the form of a clot retrieval device 2401. A distal segment 2407 of a rotatable core wire comprises a torque coil 2412 surrounding a core wire 2410. Torque coil 2412 comprises an outer coil 2413 concentrically surrounding an inner coil 2415 having windings in opposite directions. Although the coil 2412 is shown as having a constant diameter, this leaves an internal entrapped space between the coil and the core wire, as a result of the tapering core wire. When the area of the aspiration lumen between the coil and the inside wall of the corresponding catheter is optimally maximized, the diameter of the coil 2412 can taper smaller in the distal direction to track the taper of the core wire. This may be accomplished by winding the coil onto the core wire which functions as a tapered mandrel, or using other techniques known in the art. In this execution, the OD of the core wire tapers smaller in the distal direction, while the area of the aspiration lumen tapers larger in the distal direction.

The proximal end of the core wire 2410 is provided with a handle configured for manual rotation of the core wire. The handle may be a molded part such as a knob, having axially oriented surface structures such as ridges, flats or grooves to enhance grip. The handle may have two opposing tabs extending in opposite directions from the axis of the core wire, and can be rotated using two or three fingers.

As illustrated further in FIGS. 1E and 1F, the torque coil 2412 extends between a proximal end 430 and a distal end 432. The proximal end 430 is secured to a tapered portion of the core wire 2410. As illustrated in FIG. 1E, the core wire 2410 tapers from a larger diameter in a proximal zone to a smaller diameter in a distal zone 434 with a distal transition 436 between the tapered section and the distal zone 434 which may have a substantially constant diameter throughout. The inside diameter of the inner coil 2415 is complementary to (approximately the same as) the outside diameter at the proximal end 430 of the core wire 2410. The tapered section of the core wire 2410 extends proximally from the distal transition 436 to a proximal transition (not illustrated) proximal to which the core wire 2410 has a constant diameter.

The torque coil 2412 may additionally be provided with a proximal radiopaque marker and/or connector such as a solder joint 438. In the illustrated implementation, the proximal connector 438 is in the form of an annular silver solder band, surrounding the inner coil 415 and abutting a proximal end of the outer coil 2413.

The axial length of the torque coil 2412 is within the range of from about 10 mm to about 50 mm and in some embodiments within the range of from about 20 mm to about 40 mm. The distal transition 436 and the distal stopper 2414 may be positioned within the range of from about 5 mm to about 20 mm and in some implementations within the range of from about 8 mm to about 12 mm from the proximal end of the distal cap 2420.

Referring to FIG. 1E-1I, the distal stopper 2414 may be provided with one or two or three or more spokes 440, extending radially outwardly from the outer coil 413, and optionally supported by an annular hub 442 carried by the torque coil 2412. The spoke 440 supports a slider 441 having a peripheral surface 442, configured for a sliding fit within the inside diameter of the delivery catheter lumen. Preferably at least three or four or five or more spokes 440 are provided, spaced apart equidistantly to provide rotational balance. In the illustrated embodiment, three spokes 440 are provided, spaced at approximately 120° intervals around the circumference of the torque coil 2412.

In the illustrated embodiment, the spokes or struts 440 have an axial length that is greater than their width measured in the circumferential direction, and extend parallel to the longitudinal axis of the catheter. Alternatively, the spokes 440 can be oriented in a spiral configuration, to create a propeller which during rotation may assist in transport of material in the proximal direction. The leading edge may be sharpened, to sever thrombus engaged by rotation of the core wire while simultaneously applying vacuum to the central lumen.

The distal stopper 2414 carries a plurality of distal surfaces 446, such as on the slider 441. The distal surface 446 is configured to slidably engage a proximal surface of a stop on the inside diameter of the delivery catheter, such as a proximally facing surface 2405 on a radially inwardly extending annular flange or ring 2402. See FIG. 1B discussed previously. This creates a sliding interference fit with a bearing surface so that the distal stopper 2414 can rotate within the delivery catheter, and travel in an axial distal direction no farther than when distal surface 446 slideably engages the proximal surface 2405 on the stop ring 2402.

Referring to FIG. 1E, the distal end 432 of the torque coil 2412 is provided with a distal cap 2420. Distal cap 2420 may comprise an annular band such as a radiopaque marker band, bonded to the outside surface of the inner coil 2415, and axially distally adjacent or overlapping a distal end of the outer coil 2413. A proximally extending attachment such as an annular flange 2417 may be provided on the agitator tip 2416, for bonding to the distal cap 2420 and in the illustrated embodiment to the outer coil 2413. The distal cap 2420 may also be directly or indirectly bonded to a distal end of the core wire 2410.

The agitator tip 2416 is provided with a distal end 450, and a proximally extending helical flange 452 that increases in diameter in the proximal direction to a maximum diameter, and then decreases in diameter in the proximal direction to a minimum diameter that may be larger than the diameter at the distal end. The flange may extend at least about one full revolution and generally less than about five or four or three revolutions about an extension of the longitudinal axis of the core wire 2410. The helical flange is provided with a rounded, blunt edge 454, configured for slidably rotating within the tubular delivery catheter.

The maximum OD for the tip 2416 is generally at least about 0.005 inches and preferably at least about 0.01 inches or 0.015 inches or more smaller than the ID of the catheter aspiration lumen through which the embolism treatment system 2401 is intended to advance, measured at the axial operating location of the tip 2416 when the stopper 2414 is engaged with the stop ring. For example, a tip having a maximum OD in the range of from about 0.050-0.056 inches will be positioned within a catheter having a distal ID within the range of from about 0.068 to about 0.073 inches, and in one embodiment about 0.071 inches. With the tip centered in the lumen of the delivery (aspiration) catheter, the tip is spaced from the inside wall of the catheter by a distance in all directions of at least about 0.005 inches and in some embodiments at least about 0.007 inches or 0.010 inches or more.

Thus an unimpeded flow path is created in the annular space between the maximum OD of the tip, and the ID of the catheter lumen. This annular flow path cooperates with the vacuum and helical tip to grab and pull obstructive material into the catheter under rotation and vacuum. The annular flow path is significantly greater than any flow path created by manufacturing tolerances in a tip configured to shear embolic material between the tip and the catheter wall.

Additional aspiration volume is obtained as a result of the helical channel defined between each two adjacent threads of the tip. A cross sectional area of the helical flow path of a tip having a maximum OD in the range of from about 0.050 to about 0.056 inches will generally be at least about 0.0003 square inches, and in some embodiments at least about 0.00035 or at least about 0.000375 inches. The total aspiration flow path across the helical tip is therefore the sum of the helical flow path through the tip and the annular flow path defined between the OD of the tip and the ID of the catheter lumen.

The combination of a rounded edge 454 on the thread 452, slow, manual rotation of the tip through less than about 20 or 10 or 5 or less rotations, and space between the thread 452 and catheter inside wall enables aspiration both through the helical channel formed between adjacent helical threads as well as around the outside of the tip 2416 such that the assembly is configured for engaging and capturing embolic material but not shearing it between a sharp edge and the inside wall of the catheter. Once engaged, additional rotation draws the aspiration catheter distally over the clot to ensconce a proximal portion of the clot to facilitate proximal retraction and removal. The axial length of the tip 2416 including the attachment sleeve 2417 is generally less than about 6 mm, and preferably less than about 4 mm or 3 mm or 2.5 mm or less depending upon desired performance.

The pitch of the thread 452 may vary generally within the range of from about 35 degrees to about 80 degrees, depending upon desired performance. Thread pitches within the range of from about 40-50 degrees may work best for hard clots, while pitches within the range of from about 50 to 70 degrees may work best for soft clots. For some implementations the pitch will be within the range of from about 40-65 degrees or about 40-50 degrees.

The tip 2416 may additionally be provided with a feature for attracting and/or enhancing adhesion of the clot to the tip. For example, a texture such as a microporous, microparticulate, nanoporous or nanoparticulate surface may be provided on the tip, either by treating the material of the tip or applying a coating. A coating of a clot attracting moiety such as a polymer or drug may be applied to the surface of the tip. For example, a roughened Polyurathane (Tecothane, Tecoflex) coating may be applied to the surface of at least the threads and optionally to the entire tip. The polyurethane may desirably be roughened such as by a solvent treatment after coating, and adhesion of the coating to the tip may be enhanced by roughening the surface of the tip prior to coating.

Alternatively, the core wire 2410 may be provided with an insulating coating to allow propagation of a negative electric charge to be delivered to the tip to attract thrombus. Two conductors may extend throughout the length of the body, such as in a coaxial configuration. Energy parameters and considerations are disclosed in U.S. Pat. No. 10,028,782 to Orion and US patent publication No. 2018/0116717 to Taff et al., the disclosures of each of which are hereby expressly incorporated by reference in their entireties herein. As a further alternative, the tip 2416 can be cooled to cryogenic temperatures to produce a small frozen adhesion between the tip and the thrombus. Considerations for forming small cryogenic tips for intravascular catheters are disclosed in US patent publication Nos. 2015/0112195 to Berger et al., and 2018/0116704 to Ryba et al., the disclosures of each of which are hereby expressly incorporated by reference in their entireties herein.

Referring to FIG. 1G, there is illustrated a cross section through a distal stopper 2414 in which the slider 441 is a continuous circumferential wall having a continuous peripheral bearing surface 442. Three struts 440 are spaced apart to define three flow passageways 443 extending axially therethrough. The sum of the surface areas of the leading edges of the struts 440 is preferably minimized as a percentage of the sum of the surface areas of the open flow passageways 443. This allows maximum area for aspiration while still providing adequate support axially for the distal surface 446 (see FIG. 1F) to engage the complementary stop surface on the inside wall of the catheter and prevent the tip 2416 from advancing distally beyond a preset relationship with the catheter. The sum of the leading (distal facing) surface area of the struts is generally less than about 45% and typically is less than about 30% or 25% or 20% of the sum of the areas of the flow passageways 443.

In an embodiment having a torque coil 2412 with an OD of about 0.028 inches, the OD of the stopper 2414 is about 0.068 inches. The wall thickness of the struts is generally less than about 0.015 inches and typically less than about 0.010 inches and in some implementations less than about 0.008 inches or 0.005 inches or less. The struts 440 have a length in the catheter axial direction that is sufficient to support the assembly against distal travel beyond the catheter stop ring, and may be at least about 50% of the OD of the stopper 2414. In a stopper 2414 having an OD of about 0.68 inches, the struts 2440 have an axial length of at least about 0.75 mm or 0.95 mm.

Referring to FIG. 1H, there is illustrated a stopper 2414 having three distinct sliders 441 each supported by a unique strut 440. The sum of the circumference of the three peripheral surfaces is preferably no more than about 75% and in some implementations no more than about 50% or 40% of the full circumference of a continuous circumferential peripheral surface 442 as in FIG. 1G. This further increases the cross sectional area of the flow paths 443. In a catheter having an ID of no more than about 0.07 inches, an OD of the hub 443 of at least about 0.026 or 0.028 or 0.030 or more, the sum of the flow paths 443 is at least about 0.0015 inches, and preferably at least about 0.020 or 0.022 inches or more. The area of the leading edges of the struts 440 and sliders 441 is preferably less than about 0.003 inches, and preferably less than about 0.001 inches or 0.0008 inches or less. In the catheter axial direction, the length of the struts 440 is at least about 0.50 mm or 0.75 mm, and in one embodiment the length of the struts 440 and sliders 441 is about 1 mm.

One method for using the system described above is described below. An 0.088 LDP guide catheter is introduces and if possible, advanced until catheter tip is slightly proximal to occlusion site. An 0.071 aspiration catheter of FIGS. 1A and 1B is introduced through the 088 LDP and advanced until the catheter tip reaches the clot face. Any intermediate catheters or guide wires, if applicable, are removed. The rotatable core wire is introduced so that its distal tip is flush with the 0.071 aspiration catheter's distal end. Seal the proximal RHV and apply vacuum to the 0.071 aspiration catheter using an aspiration pump. The core wire is manually rotated between about 2 and 10 times, generally no more than 20 times to engage the clot without cutting, and to draw the catheter distally partially over the clot and rotation of the core wire is discontinued.

The aspiration catheter is at this point anchored to the clot using both vacuum and mechanical engagement. The 0.088 LDP catheter is then advanced over the aspiration catheter which functions like a guidewire, until the 0.088 catheter reaches the face of the clot. Vacuum is applied to the 088 LDP guide catheter using a vacuum source such as a VacLok syringe. The aspiration catheter with clot secured on its tip, is proximally retracted through the 088 LDP guide, while maintaining position of the 088 LDP at the occlusion site.

If flow has not been restored through the 088 LDP, the core wire may be removed from the aspiration catheter. If necessary, the helical tip of the core wire may be wiped to remove residual clot, and the core wire and aspiration catheter returned to the occlusion site to repeat the clot retrieval sequence until flow is restored. Once flow is restored, remove the 0.088 LDP guide catheter.

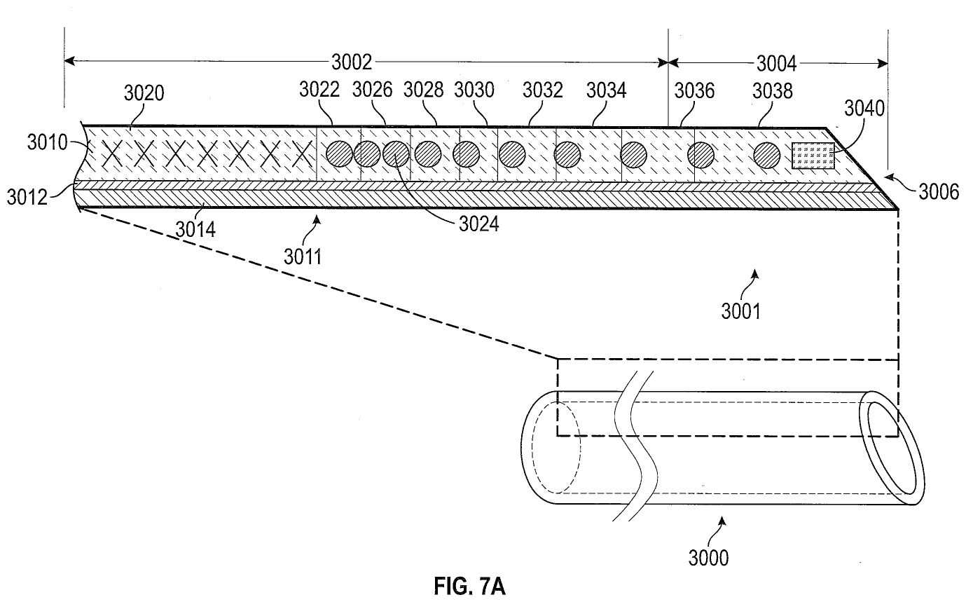

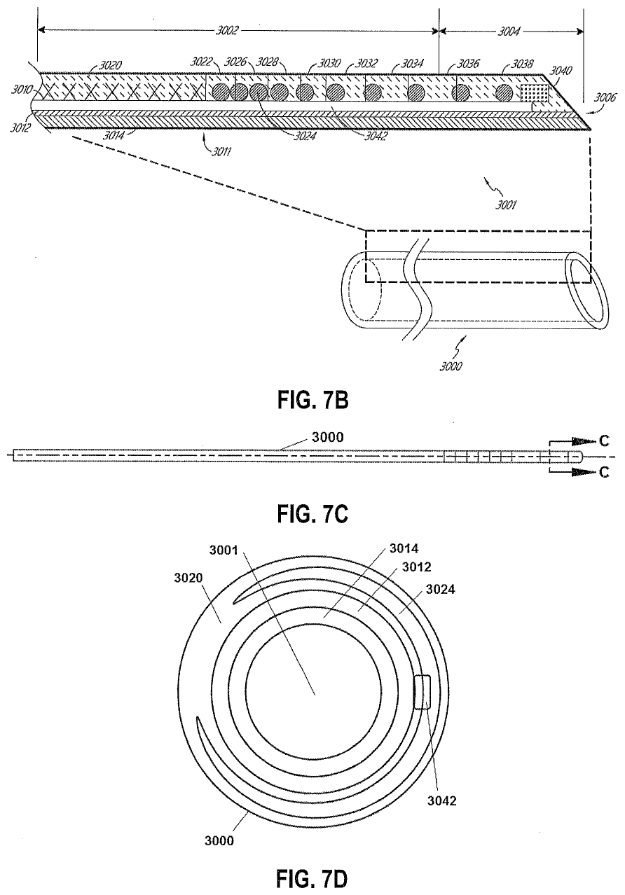

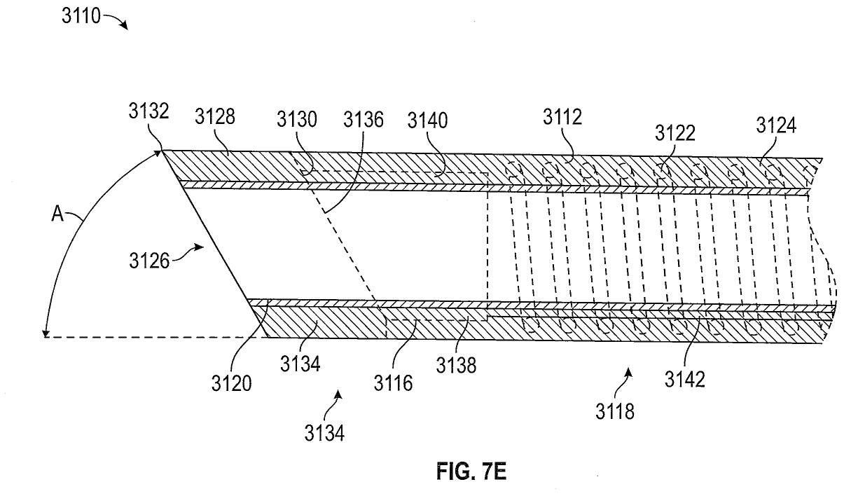

In accordance with another aspect of the invention, there is provided a torque transmission system for changing the rotational orientation of the distal end of a highly flexible catheter shaft. Certain high flexibility catheters have an angled cut and elliptical opening at the tip, discussed further for example in connection with FIG. 7E. One feature of the angled tip is to aid in navigation and aspiration of clot. Ideally, this angled cut would be oriented so that the large oval opening created is oriented to face the clot as directly as possible, even in vasculature with bends and branches. Generally, neurovascular catheters and particularly the distal wall constructions disclosed herein exhibit poor torque transmission, so that rotation of the proximal end of the catheter does not predictably provide an equivalent degree of rotation at the distal end, due to the flexible construction and thin walls. So orienting the angled tip by attempting to directly torque the catheter from the proximal end is not practical.

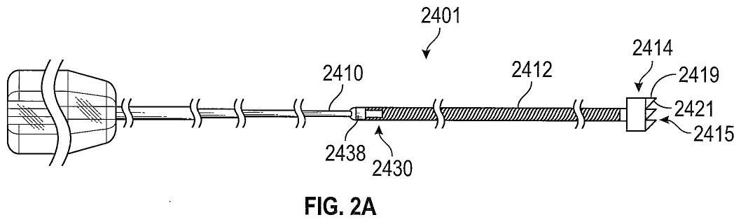

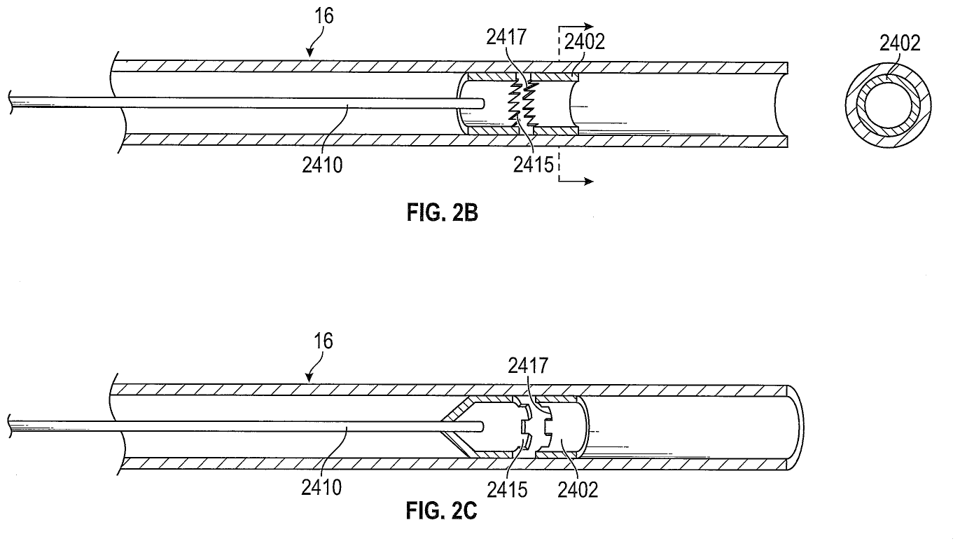

Referring to FIG. 2A, a torque wire is provided for directly transmitting torque to a distal zone of the catheter. A distal stopper 2414 such as a circular ring is attached to the core wire in a similar manner as discussed in connection with FIG. 1C. The portion of the core wire 2410 extending beyond about the distal transition 436 (See FIG. 1E) may be omitted, leaving the distal stopper 2414 at the distal end of the torque wire. Alternatively, a short lead segment of the core wire may extend through and distally beyond the distal stopper 2414 as a centering guide. The distal stopper 2414 carries a distal face carrying at least one and generally at least two or three or more engagement elements 2415 for rotationally engaging complementary engagement structures within the catheter lumen.

Referring to FIGS. 2B and 2C, a catheter tubular body 16 carries a distal restraint 2402 as previously discussed. A proximal surface includes at least a second engagement structure 2417 for rotationally engaging at least a first engagement structure 2415 on the distal surface of the distal stopper. In FIGS. 2A and 2B, the engagement structures are complementary interlocking ramped teeth to allow the distal face 2415 formed into the distal end of the torquable guide wire and the proximal face of the distal restraint 2402 to engage and be keyed together. This provides a mechanical linkage to transmit torque to rotate the catheter in response to rotation of the wire.

In the embodiment of FIGS. 2A and 2B, the engagement elements comprise axially distally extending projections having a first surface 2419 extending substantially in parallel with a longitudinal axis of the wire, and an opposing inclined surface 2421 which extends at an inclined angle of at least about 15 or 20 or more degrees relative to the longitudinal axis. The complementary projections and intervening recesses on the stop ring engage when the wire is rotated in a first direction to responsively rotate the stop ring, but may skip over each other when the wire is rotated in a second, opposite direction so that the catheter is not rotated in response to rotation of the wire.

The construct of FIG. 2B may be integrated into the system of any of FIGS. 1A-1H. This allows rotation of the wire in a first direction to rotate the catheter to a desired rotational position within the vessel and adjacent the obstruction. The wire may then be rotated in the second, opposite direction to rotate the tip 2416 and engage the obstruction to facilitate removal as has been previously described.

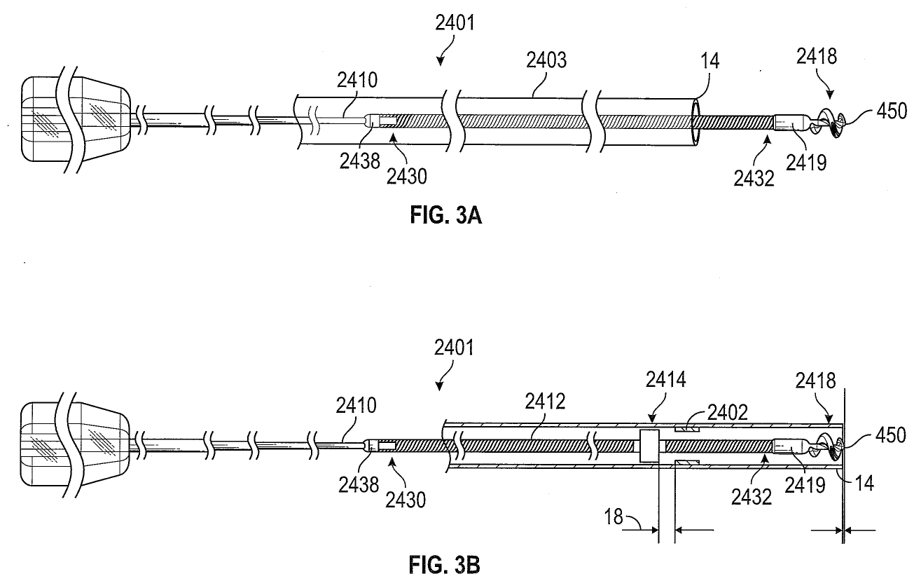

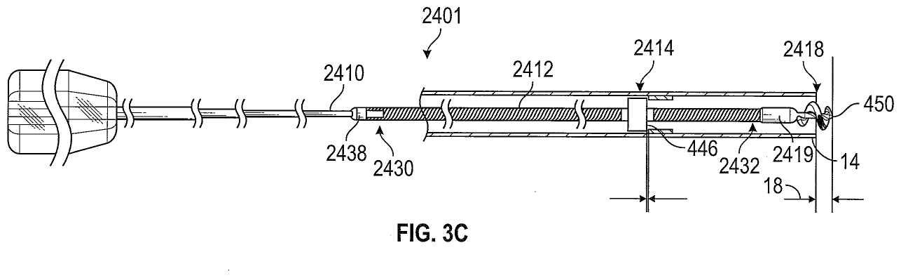

These complementary engagement surfaces may alternatively be bi-directional to engage in either rotational direction, such as at least one projection on the wire for engaging at least one recess on the catheter. In the illustrated embodiment, the engagement structures 2415 comprise at least one a square edged tooth for rotationally engaging a corresponding recess such as an axially extending slot. (See, e.g., FIG. 3B). This permits coupling and rotation of the catheter in either direction.

Alternatively, either one or both of the complementary engagement surfaces carried by the distal stopper 2414 and the distal restraint 2402 may be provided with a friction enhancing feature such as a textured surface or a material or coating having a sufficiently elevated stiction to transfer sufficient torque from the core wire to the catheter body to rotationally redirect the tip of the catheter, such as to enter or avoid a side branch, or present a different relationship between the catheter and an embolic material.

When the clot and catheter angular orientation (see FIG. 7E) is not favorable for presenting the largest opening of the catheter directly towards the clot, the wire is advanced against the catheter's internal stop ring and the complementary engagement surfaces engage so that the wire can be used to provide torque to rotationally reorient the catheter tip more favorably for clot aspiration.

In addition, the catheter tip may be rotated during distal advance of the catheter through tortuous vasculature, to optimize the angular orientation of the angled tip to facilitate distal advance of the catheter. Thus the catheter angled tip can be accurately rotated to avoid getting caught on arterial branches or calcifications.

In an implementation illustrated in FIG. 3A, a torque wire such as a clot retrieval device 2401 is similar to that illustrated in FIG. 1C, except without the distal stopper 2414. In this implementation, the clot retrieval device can be freely axially advanceable through the lumen of, and beyond the distal end of an outer catheter 2403. The clot retrieval device 2401 can be distally advanced to engage a clot, and the outer catheter 2403 distally advanced over the wire with traction on the clot retrieval device and optionally with the addition of vacuum through the catheter 2403 to grasp and remove the clot.

Alternatively, the helical tip 450 can be rotated into and optionally through the obstruction clot to act as an anchor on a wire. The catheter 2403 may be proximally withdrawn, leaving the anchored core wire 2410 in place. The core wire 2410 can then be used as a guide wire, to guide other interventional devices over or along the wire to reach the obstruction to perform additional functions. The core wire 2410 may be provided with an elongate central lumen extending between a proximal port at the proximal end, and a distal port at the distal tip 450. Following placement of the distal tip, a guidewire may be advanced through the central lumen and through the occlusion if the distal tip 450 has been rotated that far distally. The tip 450 and core wire 2410 may then be reverse rotated to disengage from the obstruction, and proximally withdrawn from the patient, leaving the guidewire in place for a subsequent procedure.

In an alternate configuration, the catheter 2403 may be provided with a second lumen such as for irrigation, aspiration, or receiving a guidewire therethrought, extending axially from a proximal port to a distal opening at or near the distal end 14. At a desired point in the procedure, the helix and catheter 2403 may be proximally withdrawn, leaving the guidewire in place for further access.

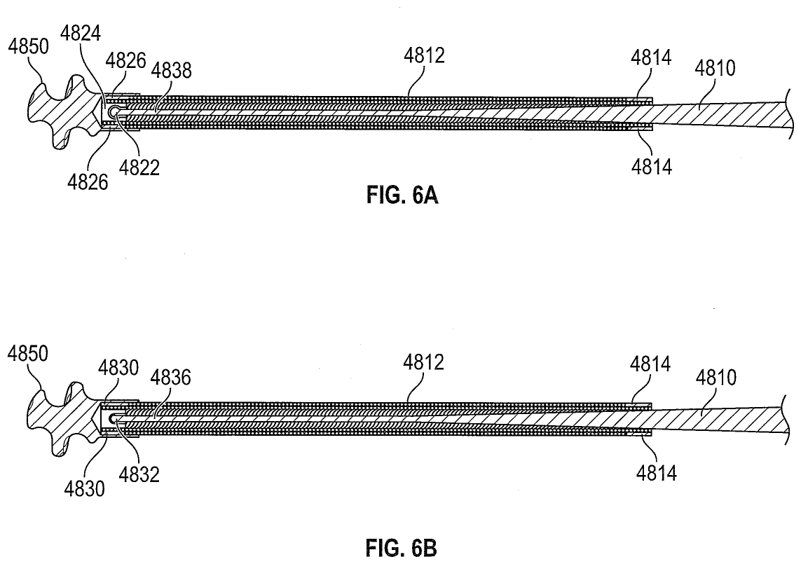

In the implementation illustrated in FIGS. 3B and 3C, the torque wire and complementary catheter are configured in a manner that is similar to FIGS. 1A-1H, except the distal tip 450 is permitted to extend a small, controlled distance beyond the distal end 14 of the catheter 10. In FIG. 3B, distal tip 450 is approximately axially aligned with the distal end 14 of catheter 10 (or distal tip 3132 in FIG. 6E). This leaves an axial gap 18 between the complementary stop surfaces on the axial restraint 2404 and the distal stopper 2414. As illustrated in FIG. 3C, bringing the complementary stop surfaces into contact allows the distal tip 450 to extend outside of the catheter by a maximum of the distance 18. Distance 18 may be at least about 0.5 mm or about 1 mm or 2 mm, but generally no more than about 1.5 cm or 1 cm or 0.5 cm depending upon the desired functionality. In some embodiments, the distance 18 is within the range of from about 0.5-3 mm.

Alternatively, the maximum distal extension distance 18 may be related to the pitch of the helical thread. For example, 18 may be a distance equivalent to the axial length of from about one to about five revolutions of the thread, and preferably within the range of from about one to about three revolutions of the thread.

In use, the distal tip 450 of the system of FIGS. 3B and 3C can be extended beyond the catheter and rotated to engage the occlusion. As the helix is rotated, it pulls the catheter forward (distally) and allows the helix to advance further distally over the core wire.

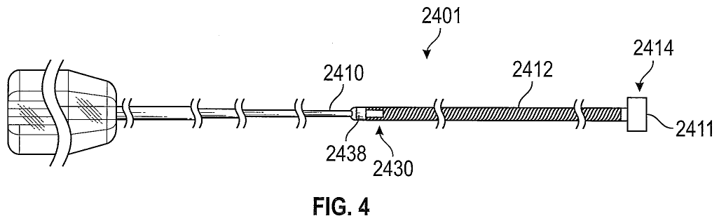

A further application of the distal stopper on a wire construction is illustrated in FIG. 4. The core wire 2410 extends only as far as about the transition 436 (FIG. 1E) so the distal stopper resides at the distal end of the wire. A distal face 2411 on the distal stopper 2414 can be used to tap against the proximal surface 2405 on a distal restraint 2402 (see FIG. 1B) to provide a jack hammer effect, pulling the catheter from the distal end rather than pushing it from the proximal end. Tapping may be low frequency, accomplished manually by the clinician, or higher frequency such as at least about 10 Hz or 100 Hz or ultrasound, depending upon the catheter construction and desired clinical result.

This construct may lower the column strength requirement along the length of the catheter body, at least from a pushability perspective. Maintaining hoop strength is still desirable in a catheter intended to be placed under vacuum. But in a non vacuum device the sidewall of the catheter may be able to be reduced if the target site may be reached by distal ‘pulling’ the catheter from the distal restraint 2402 rather than pushing from the proximal manifold.

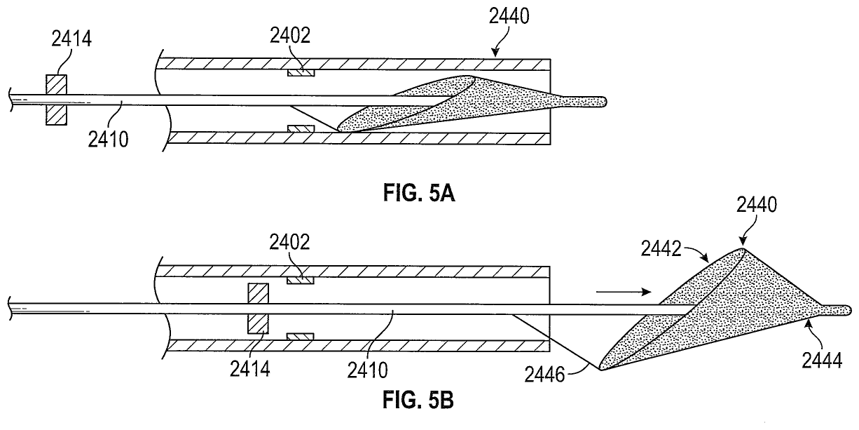

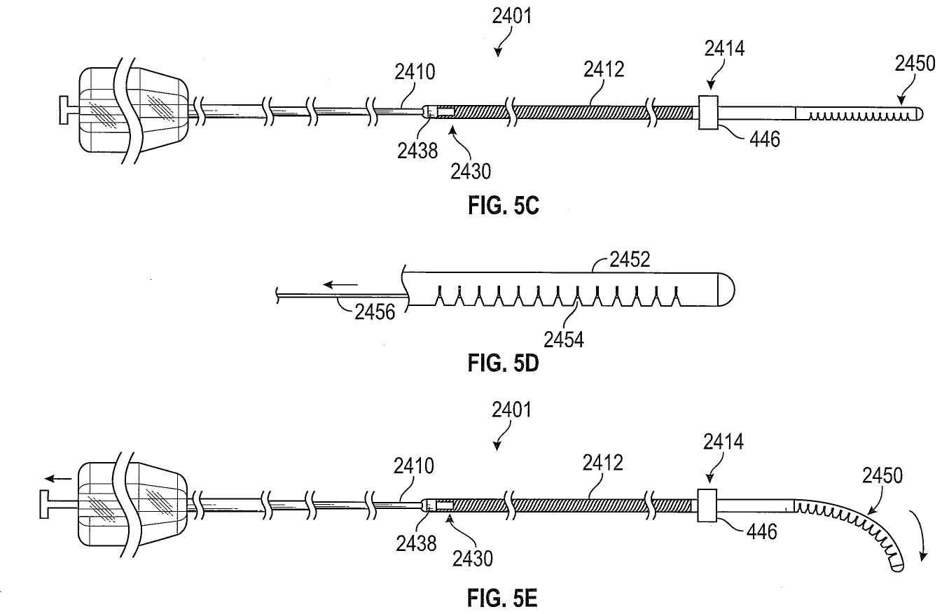

Referring to FIGS. 5A and 5B, there are illustrated side elevational views of a blood flow assisted access wire for use with a catheter having a stop ring. The wire 2410 carries a stopper 2414, for limiting distal travel of the wire relative to the catheter by an interference fit with stop ring 2402, as has been discussed. A force transfer element 2440 is carried by the wire 2410, and configured to move between a radially collapsed configuration for positioning within the catheter (FIG. 5A), and a radially enlarged configuration when advanced distally out of the catheter (FIG. 5B). The force transfer element 2440 is adapted to at least partially obstruct blood flow, and transfer distally directed force to the wire 2410. Responsive distal downstream advance of the force transfer element 2440 causes the stopper 2414 to engage the stop ring 2402, and pull the catheter in an antegrade direction.

In the illustrated implementation, the force transfer element 2440 comprises a conical membrane such as a filter having an open proximal end 2442 and a closed distal end 2444. The proximal opening 2442 may be supported by a nitinol wire loop, which is connected to the wire 2410 by way of an inclined strut 2446. Inclined strut 2446 facilitates reentry of the force transfer element 2440 into the distal end of the catheter, upon proximal withdrawal of the wire 2410.

In an alternate execution of the present invention, the force transfer element 2440 may comprise an alternate structure for capturing force from blood flow, including an inflatable balloon. The wire 2410 may be provided with a central lumen, extending throughout its length, and in communication with the balloon, to accomplish the inflation and deflation as is understood in the art.

Referring to FIGS. 5C-5E there is illustrated a wire 2410 with a steerable distal region 2450. The steering region 2450 may comprise a tubular body having a first side 2452 which is relatively axially non-collapsible. A second, typically opposing side is provided with a plurality of transverse slots 2454 which permit axial collapse. A pull wire 2456 is attached to a distal end of the steering zone 2450. Proximal retraction of the pull wire 2456 relative to the tubular body causes axial collapse of the transverse slots 2454 and resulting curvature as shown in FIG. 5E.

Any of the catheter shaft or sections of the catheter shaft in accordance with the present invention may comprise a multi-layer construct having a high degree of flexibility and sufficient push ability to reach deep into the cerebral vasculature, such as at least as deep as the petrous, cavernous, or cerebral segment of the internal carotid artery (ICA).

In one example, referring to FIG. 7A, the catheter 3000 may have an effective length from the manifold to distal tip from about 70 cm to about 150 cm, from about 80 cm to about 140 cm, from about 90 cm to about 130 cm, from about 100 cm to about 120 cm, or from about 105 cm to about 115 cm. The outer diameter of the catheter 3000 may be from about 0.07 inches to about 0.15 inches, from about 0.08 inches to about 0.14 inches, from about 0.09 inches to about 0.13 inches, from about 0.1 inches to about 0.12 inches, or from about 0.105 inches to about 0.115 inches, and may be lower in a distal segment than in a proximal segment. The inner diameter 3108 of the catheter 3000 in a single central lumen embodiment may be greater than or equal to about 0.11 inches, greater than or equal to about 0.1 inches, greater than or equal to about 0.09 inches, greater than or equal to about 0.088 inches, greater than or equal to about 0.08 inches, greater than or equal to about 0.07 inches, greater than or equal to about 0.06 inches, or greater than or equal to about 0.05 inches. The inner diameter 3108 of the catheter 3000 in a single central lumen embodiment may be less than or equal to about 0.11 inches, less than or equal to about 0.1 inches, less than or equal to about 0.09 inches, less than or equal to about 0.088 inches, less than or equal to about 0.08 inches, less than or equal to about 0.07 inches, less than or equal to about 0.06 inches, or less than or equal to about 0.05 inches.

Referring to FIG. 7A, an inner liner 3014 may be formed by dip coating a mandrel (not shown) to provide a thin walled tubular inside layer of the catheter body 3000. The dip coating may be produced by coating a wire such as a silver coated copper wire in PTFE. The mandrel may thereafter be axially elongated to reduce diameter, and removed to leave the tubular inner liner.

The outside surface of the tubular inner liner 3014 may thereafter be coated with a soft tie layer 3012 such as polyurethane (e.g., Tecoflex™), to produce a layer having a thickness of no more than about 0.005 inches, and in some implementations approximately 0.001 inches. The tie layer 3012 will generally extend along at least about the most distal 10 cm or 20 cm of the catheter shaft 3000 generally less than about 50 cm and may in one implementation extend approximately the distal 30 cm of the catheter shaft 3000, 3100.

A braid such as a 75 ppi stainless steel braid 3010 may thereafter be wrapped around the inner liner 3014 through a proximal zone up to a distal transition 3011. From the distal transition 3011 to the distal end of the catheter 3000, a coil 3024 comprising a shape memory material such as a Nitinol alloy may thereafter be wrapped around the inner liner 3014. In one implementation, the Nitinol coil has a transition temperature below body temperature so that the Nitinol resides in the austinite (springy) state at body temperature. Adjacent loops or filars of the coil 3024 may be closely tightly wound in a proximal zone with a distal section having looser spacing between adjacent loops. In an embodiment having a coil section 3024 with an axial length of at least between about 20% and 30% of the overall catheter length, (e.g., 28 cm coil length in a 110 cm catheter shaft 3000), at least the distal 1 or 2 or 3 or 4 cm of the coil will have a spacing that is at least about 130%, and in some implementations at least about 150% or more than the spacing in the proximal coil section. In a 110 cm catheter shaft 3000 having a Nitinol coil the spacing in the proximal coil may be about 0.004 inches and in the distal section may be at least about 0.006 inches or 0.007 inches or more.

In embodiments comprising an extension catheter, the distal extendable section of the catheter may be constructed according to the foregoing. The length of the coil 3024 may be proportioned to the length of the extendable catheter segment or the total (e.g., extended) length of the catheter 3000. The coil 3024 may extend from a distal end of the extendable segment over at least about 50%, 60%, 70%, 80%, or 90% of the length of the extendable segment. In some embodiments, the catheter 3000 or the extendable segment may not comprise a braid and the coil 3024 may extend to the proximal end of the extendable segment (100% of the length).

The distal end of the coil 3024 can be spaced proximally from the distal end of the inner liner 3014, for example, to provide room for an annular radiopaque marker 3040. The coil 3024 may be set back proximally from the distal end, in some embodiments, by approximately no more than 1 cm, 2 cm, or 3 cm. In one embodiment, the distal end of the catheter 3000 is provided with a beveled distal surface 3006 residing on a plane having an angle of at least about 10° or 20° and in one embodiment about 30° with respect to a longitudinal axis of the catheter 3000. The radiopaque marker 3040 may reside in a plane that is transverse to the longitudinal axis. Alternatively, at least the distally facing edge of the annular radiopaque marker 3040 may be an ellipse, residing on a plane which is inclined with respect to the longitudinal axis to complement the bevel angle of the distal surface 3006. Additional details are described in connection with FIG. 7E below.

After applying the proximal braid 3010, the distal coil 3024 and the RO marker 3040 an outer Jacket 3020 maybe applied such as a shrink wrap tube to enclose the catheter body 3000. The outer shrink-wrapped sleeve 3020 may comprise any of a variety of materials, such as polyethylene, polyurethane, polyether block amide (e.g., PEBAX™) nylon or others known in the art. Sufficient heat is applied to cause the polymer to flow into and embed the proximal braid and distal coil.

In one implementation, the outer shrink wrap jacket 3020 is formed by sequentially advancing a plurality of short tubular segments 3022, 3026, 3028, 3030, 3032, 3034, 3036, 3038 concentrically over the catheter shaft subassembly, and applying heat to shrink the sections on to the catheter 3000 and provide a smooth continuous outer tubular body. The foregoing construction may extend along at least the most distal 10 cm, and preferably at least about the most distal 20 cm, 25 cm, 30 cm, 35 cm, 40 cm, or more than 40 cm of the catheter body 3000. The entire length of the outer shrink wrap jacket 3020 may be formed from tubular segments and the length of the distal tubular segments (e.g., 3022, 3026, 3028, 3030, 3032, 3034, 3036, 3038) may be shorter than the one or more tubular segments forming the proximal portion of the outer shrink wrap jacket 3020 in order to provide steeper transitions in flexibility toward the distal end of the catheter 3000.

The durometer of the outer wall segments may decrease in a distal direction. For example, proximal segments such as 3022 and 3026, may have a durometer of at least about 60 or 70D, with gradual decrease in durometer of successive segments in a distal direction to a durometer of no more than about 35D or 25D or lower. A 25 cm section may have at least about 3 or 5 or 7 or more segments and the catheter 3000 overall may have at least about 6 or 8 or 10 or more distinct flexibility zones. The distal 1 or 2 or 4 or more segments 3036, 3038, may have a smaller OD following shrinking than the more proximal segments 3022-3034 to produce a step down in OD for the finished catheter body 3000. The length of the lower OD section 3004 may be within the range of from about 3 cm to about 15 cm and in some embodiments is within the range of from about 5 cm to about 10 cm such as about 7 or 8 cm, and may be accomplished by providing the distal segments 3036, 3038 with a lower wall thickness.

Referring to FIGS. 7B and 7D, the catheter may further comprise a tension support for increasing the tension resistance in the distal zone. The tension support may comprise a filament and, more specifically, may comprise one or more axially extending filaments 3042. The one or more axially extending filaments 3042 may be axially placed inside the catheter wall near the distal end of the catheter. The one or more axially extending filaments 3042 serve as a tension support and resist elongation of the catheter wall under tension (e.g., when the catheter is being proximally retracted through tortuous vasculature).

At least one of the one or more axially extending filaments 3042 may proximally extend along the length of the catheter wall from within about 1.0 cm from the distal end of the catheter to less than about 5 cm from the distal end of the catheter, less than about 10 cm from the distal end of the catheter, less than about 15 cm from the distal end of the catheter, less than about 20 cm from the distal end of the catheter, less than about 25 cm from the distal end of the catheter, less than about 30 cm from the distal end of the catheter, less than about 35 cm from the distal end of the catheter, less than about 40 cm from the distal end of the catheter, or less than about 50 cm from the distal end of the catheter.

The one or more axially extending filaments 3042 may have a length greater than or equal to about 50 cm, greater than or equal to about 40 cm, greater than or equal to about 35 cm, greater than or equal to about 30 cm, greater than or equal to about 25 cm, greater than or equal to about 20 cm, greater than or equal to about 15 cm, greater than or equal to about 10 cm, or greater than or equal to about 5 cm.

At least one of the one or more axially extending filaments 3042 may have a length less than or equal to about 50 cm, less than or equal to about 40 cm, less than or equal to about 35 cm, less than or equal to about 30 cm, less than or equal to about 25 cm, less than or equal to about 20 cm, less than or equal to about 15 cm, less than or equal to about 10 cm, or less than or equal to about 5 cm. At least one of the one or more axially extending filaments 3042 may extend at least about the most distal 50 cm of the length of the catheter, at least about the most distal 40 cm of the length of the catheter, at least about the most distal 35 cm of the length of the catheter, at least about the most distal 30 cm of the length of the catheter, at least about the most distal 25 cm of the length of the catheter, at least about the most distal 20 cm of the length of the catheter, at least about the most distal 15 cm of the length of the catheter, at least about the most distal 10 cm of the length of the catheter, or at least about the most distal 5 cm of the length of the catheter.

In some implementations, the filament extends proximally from the distal end of the catheter along the length of the coil 24 and ends proximally within about 5 cm or 2 cm or less either side of the transition 3011 between the coil 3024 and the braid 3010. The filament may end at the transition 3011, without overlapping with the braid 3010.

In another embodiment, the most distal portion of the catheter 3000 may comprise a durometer of less than approximately 35D (e.g., 25D) to form a highly flexible distal portion of the catheter and have a length between approximately 25 cm and approximately 35 cm. The distal portion may comprise one or more tubular segments of the same durometer (e.g., segment 3038). A series of proximally adjacent tubular segments may form a transition region between a proximal stiffer portion of the catheter 3000 and the distal highly flexible portion of the catheter. The series of tubular segments forming the transition region may have the same or substantially similar lengths, such as approximately 1 cm.

The relatively short length of the series of tubular segments may provide a steep drop in durometer over the transition region. For example, the transition region may have a proximal tubular segment 3036 (proximally adjacent the distal portion) having a durometer of approximately 35D. An adjacent proximal segment 3034 may have a durometer of approximately 55D. An adjacent proximal segment 3032 may have a durometer of approximately 63D. An adjacent proximal segment 3030 may have a durometer of approximately 72D.

More proximal segments may comprise a durometer or durometers greater than approximately 72D and may extend to the proximal end of the catheter or extension catheter segment. For instance, an extension catheter segment may comprise a proximal portion greater than approximately 72D between about 1 cm and about 3 cm. In some embodiments, the proximal portion may be about 2 cm long. In some embodiments, the most distal segments (e.g., 3038-3030) may comprise PEBAX™ and more proximal segments may comprise a generally stiffer material, such as Vestamid®.

The inner diameter of the catheter 3000 or catheter extension segment may be between approximately 0.06 and 0.08 inches, between approximately 0.065 and 0.075 inches, or between 0.068 and 0.073 inches. In some embodiments, the inner diameter is approximately 0.071 inches.

In some embodiments, the distal most portion may taper to a decreased inner diameter as described elsewhere herein. The taper may occur approximately between the distal highly flexible portion and the transition region (e.g., over the most proximal portion of the distal highly flexible portion). The taper may be relatively gradual (e.g., occurring over approximately 10 or more cm) or may be relatively steep (e.g., occurring over less than approximately 5 cm). The inner diameter may taper to an inner diameter between about 0.03 and 0.06 inches. For example, the inner diameter may be about 0.035 inches, about 0.045 inches, or about 0.055 inches at the distal end of the catheter 3000. In some embodiments, the inner diameter may remain constant, at least over the catheter extension segment.

In some embodiments, the coil 3024 may extend proximally from a distal end of the catheter 3000 along the highly flexible distal portion ending at the distal end of the transition region. In other embodiments, the coil 3024 may extend from a distal end of the catheter to the proximal end of the transition region, to a point along the transition region, or proximally beyond the transition region. In other embodiments, the coil 3024 may extend the entire length of the catheter 3000 or catheter extension segment as described elsewhere herein. The braid 3010, when present, may extend from the proximal end of the coil 3024 to the proximal end of the catheter 3000 or catheter extension segment.

The one or more axially extending filaments 3042 may be placed near or radially outside the tie layer 3012 or the inner liner 3014. The one or more axially extending filaments 3042 may be placed near or radially inside the braid 3010 and/or the coil 3024. The one or more axially extending filaments 3042 may be carried between the inner liner 3014 and the helical coil 3024.

When more than one axially extending filaments 3042 are placed in the catheter wall, the axially extending filaments 3042 may be placed in a radially symmetrical manner. For example, the angle between the two axially extending filaments 3042 with respect to the radial center of the catheter may be about 180 degree. Alternatively, depending on desired clinical performances (e.g., flexibility, trackability), the axially extending filaments 3042 may be placed in a radially asymmetrical manner. The angle between any two axially extending filaments 3042 with respect to the radial center of the catheter may be less than about 180 degree, less than or equal to about 165 degree, less than or equal to about 150 degree, less than or equal to about 135 degree, less than or equal to about 120 degree, less than or equal to about 105 degree, less than or equal to about 90 degree, less than or equal to about 75 degree, less than or equal to about 60 degree, less than or equal to about 45 degree, less than or equal to about 30 degree, less than or equal to about 15 degree, less than or equal to about 10 degree, or less than or equal to about 5 degree.

The one or more axially extending filaments 3042 may be made of materials such as Kevlar, Polyester, Meta-Para-Aramide, or any combinations thereof. At least one of the one or more axially extending filaments 3042 may comprise a single fiber or a multi-fiber bundle, and the fiber or bundle may have a round or rectangular cross section. The terms fiber or filament do not convey composition, and they may comprise any of a variety of high tensile strength polymers, metals or alloys depending upon design considerations such as the desired tensile failure limit and wall thickness. The cross-sectional dimension of the one or more axially extending filaments 3042, as measured in the radial direction, may be no more than about 1%, 2%, 3%, 4%, 5%, 6%, 7%, 8%, 9%, 10%, 11%, 12%, 13%, 14%, 15%, 20%, 25%, or 30% of that of the catheter 3000. The cross-sectional dimension of the one or more axially extending filaments 3042, as measured in the radial direction, may be no more than about 0.001 inches, about 0.002 inches, about 0.003 inches, about 0.004 inches, about 0.005 inches, about 0.006 inches, about 0.007 inches, about 0.008 inches, about 0.009 inches, about 0.010 inches, about 0.015 inches, about 0.020 inches, about 0.025 inches, or about 0.030 inches.

The one or more axially extending filaments 3042 may increase the tensile strength of the distal zone of the catheter to at least about 1 pound, at least about 2 pounds, at least about 3 pounds, at least about 4 pounds, at least about 5 pounds, at least about 6 pounds, at least about 7 pounds, at least about 8 pounds, or at least about 10 pounds or more.

Any of the aspiration catheters or tubular extension segments disclosed herein, whether or not an axial filament is included, may be provided with an angled distal tip. Referring to FIG. 7E, distal catheter tip 3110 comprises a tubular body 3112 which includes an advance segment 3114, a marker band 3116 and a proximal segment 3118. An inner tubular liner 3120 may extend throughout the length of the distal catheter tip 3110, and may comprise dip coated PTFE.

A reinforcing element 3122 such as a braid or spring coil is embedded in an outer jacket 3124 which may extend the entire length of the distal catheter tip 3110.

The advance segment 3114 terminates distally in an angled face 3126, to provide a leading side wall portion 3128 having a length measured between the distal end 3130 of the marker band 3116 and a distal tip 3132. A trailing side wall portion 3134 of the advance segment 3114, has an axial length in the illustrated embodiment of approximately equal to the axial length of the leading side wall portion 3128 as measured at approximately 180 degrees around the catheter from the leading side wall portion 3128. The leading side wall portion 3128 may have an axial length within the range of from about 0.1 mm to about 5 mm and generally within the range of from about 1 to 3 mm. The trailing side wall portion 3134 may be at least about 0.1 or 0.5 or 1 mm or 2 mm or more shorter than the axial length of the leading side wall portion 3128, depending upon the desired performance.

The angled face 3126 inclines at an angle A within the range of from about 45 degrees to about 80 degrees from the longitudinal axis of the catheter. For certain implementations, the angle is within the range of from about 55 degrees to about 65 degrees or within the range of from about 55 degrees to about 65 degrees from the longitudinal axis of the catheter. In one implementation the angle A is about 60 degrees. One consequence of an angle A of less than 90 degrees is an elongation of a major axis of the area of the distal port which increases the surface area of the port and may enhance clot aspiration or retention. Compared to the surface area of the circular port (angle A is 90 degrees), the area of the angled port is generally at least about 105%, and no more than about 130%, in some implementations within the range of from about 110% and about 125% and in one example is about 115%.

In the illustrated embodiment, the axial length of the advance segment is substantially constant around the circumference of the catheter, so that the angled face 3126 is approximately parallel to the distal surface 3136 of the marker band 3116. The marker band 3116 has a proximal surface approximately transverse to the longitudinal axis of the catheter, producing a marker band 3116 having a right trapezoid configuration in side elevational view. A short sidewall 3138 is rotationally aligned with the trailing side wall portion 3134, and has an axial length within the range of from about 0.2 mm to about 4 mm, and typically from about 0.5 mm to about 2 mm. An opposing long sidewall 3140 is rotationally aligned with the leading side wall portion 3128. Long sidewall 3140 of the marker band 3116 is generally at least about 10% or 20% longer than short sidewall 3138 and may be at least about 50% or 70% or 90% or more longer than short sidewall 3138, depending upon desired performance. Generally the long sidewall 3140 will have a length of at least about 0.5 mm or 1 mm and less than about 5 mm or 4 mm.

The marker band may have at least one and optionally two or three or more axially extending slits throughout its length to enable radial expansion. The slit may be located on the short sidewall 3138 or the long sidewall 3140 or in between, depending upon desired bending characteristics. The marker band may comprise any of a variety of radiopaque materials, such as a platinum/iridium alloy, with a wall thickness preferably no more than about 0.003 inches and in one implementation is about 0.001 inches.

The marker band zone of the assembled catheter will have a relatively high bending stiffness and high crush strength, such as at least about 50% or at least about 100% less than proximal segment 18 but generally no more than about 200% less than proximal segment 3118. The high crush strength may provide radial support to the adjacent advance segment 3114 and particularly to the leading side wall portion 3128, to facilitate the functioning of distal tip 3132 as an atraumatic bumper during transluminal advance and to resist collapse under vacuum. The proximal segment 3118 preferably has a lower bending stiffness than the marker band zone, and the advance segment 3114 preferably has even a lower bending stiffness and crush strength than the proximal segment 3118.

The advance segment 3114 may comprise a distal extension of the outer jacket 3124 and optionally the inner liner 3120, without other internal supporting structures distally of the marker band 3116. Outer jacket may comprise extruded Tecothane. The advance segment 3114 may have a bending stiffness and radial crush stiffness that is no more than about 50%, and in some implementations no more than about 25% or 15% or 5% or less than the corresponding value for the proximal segment 3118.

A support fiber 3142 as has been discussed elsewhere herein extends through at least a distal portion of the length of the proximal segment 3118. As illustrated, the support fiber 3142 may terminate distally at a proximal surface of the marker band 3116 and extend axially radially outwardly of the tubular liner 3120 and radially inwardly from the support coil 3122. Fiber 3142 may extend substantially parallel to the longitudinal axis, or may be inclined into a mild spiral having no more than 10 or 7 or 3 or 1 or less complete revolutions around the catheter along the length of the spiral. The fiber may comprise a high tensile strength material such as a multifilament yarn spun from liquid crystal polymer such as a Vectran multifilament LCP fiber.

Depending on whether the catheter 3000 is able to navigate sufficiently distally to reach the target site, an intraluminal extension catheter such as a tubular telescopic extension segment having a proximally extending control wire may be inserted through the catheter 3000 from the proximal end of the catheter 3000. The extension segment is inserted and distally advanced such that the distal end of the extension segment reaches further distally beyond the distal end of the catheter 3000. The outer diameter of the extension segment is smaller than the inner diameter of the catheter 3000. This way, the extension segment can slide inside the lumen of the catheter 3000.

The extension segment incorporates characteristics of the side wall construction of the catheter 3000 described herein. The axial length of the tubular extension segment may be less than about 50% and typically less than about 25% of the length of the catheter 3000. The axial length of the tubular extension segment will generally be at least about 10 cm or 15 cm or 20 cm or 25 cm or more but generally no more than about 70 cm or 50 cm or 30 cm.

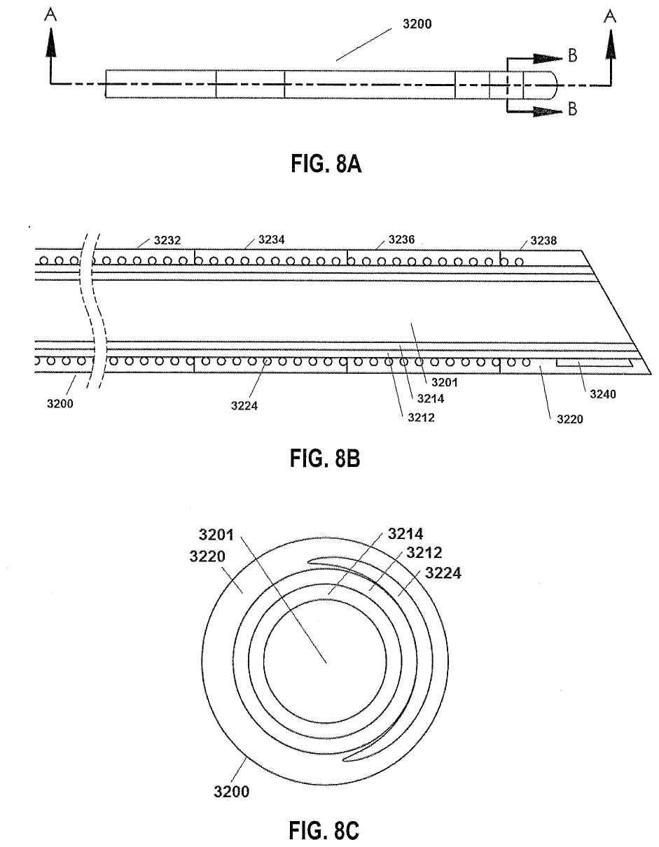

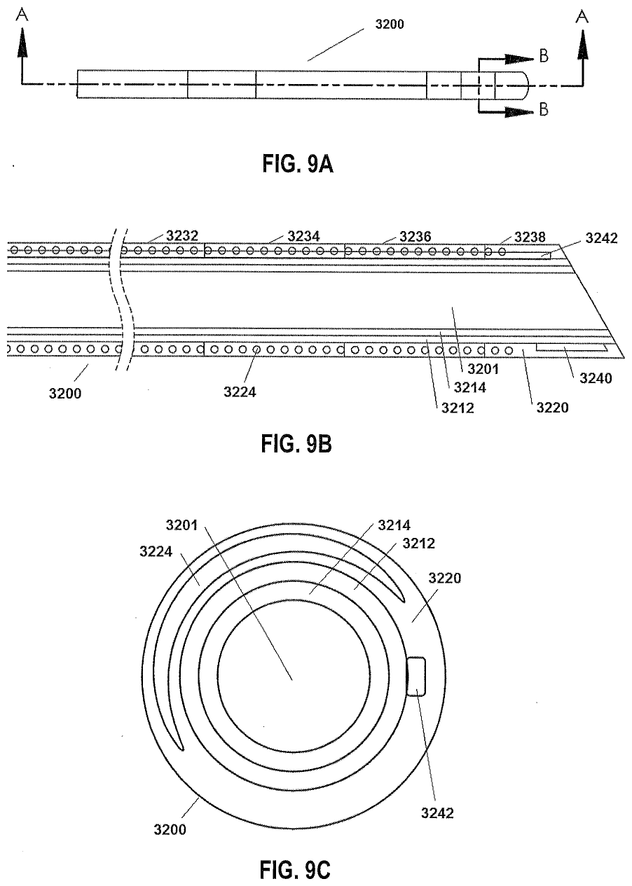

Referring to FIGS. 8A-8C, any of the catheters described herein may have one or more axially extending filaments 3242.

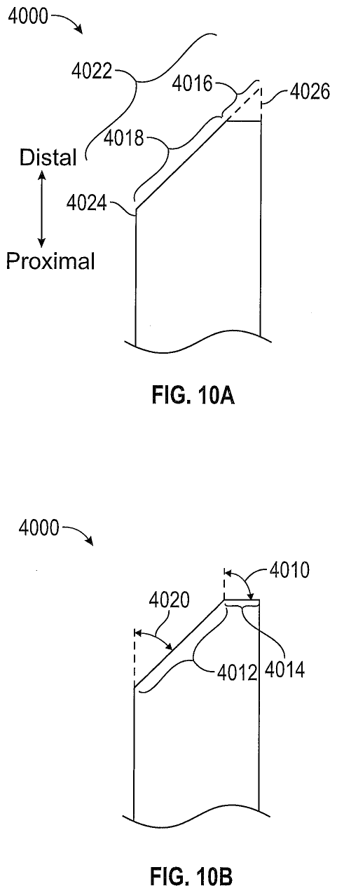

Referring to FIGS. 10A-10B, the angled face 3126 of the catheter of FIG. 7E is further modified into a blunted, angled face 4000, such that leading edge tip (shown in FIG. 7E) is removed. As shown in FIG. 10B, the catheter distal face 4000 comprises a first section 4012 that resides on a first plane which crosses a longitudinal axis of the tubular body at a first angle 4020 within the range of from about 35 degrees to about 55 degrees, and a second section 4014 that resides on second plane which crosses the longitudinal axis of the tubular body at a second angle 4010 within the range from about 55 degrees to about 90 degrees. Section 4016, shown in FIG. 10A, of distal face 4000, shown in FIG. 7E, is removed. One consequence of an angle 4020 of less than 90 degrees and a blunted length 4014 is an elongation of a major axis of the area of the distal port which increases the surface area of the port and may enhance clot aspiration or retention while also minimizing the potential for vessel wall damage.

Compared to the surface area of the circular port (angle 4020 and angle 4010 are both 90 degrees), the area of the angled blunted port is generally at least about 105%, and no more than about 150%, in some implementations within the range of from about 110% and about 125% and in one example is about 115%. The degree of blunting can be defined by a ratio of unblunted axial length 4022 measured from trailing edge 4024 to leading edge 4026 to blunted axial length shown as section 4018.

As shown in FIG. 10A, a blunting ratio of ¾ is shown. A length 4016 which tip 4000 is blunted may be 1-5 mm, 1-4 mm, 1-3 mm, 1-2 mm, 0.1-0.2 mm, 0.2-0.3 mm, 0.3-0.4 mm, 0.4-0.5 mm, 0.5-0.6 mm, 0.6-0.7 mm, 0.7-0.8 mm, 0.8-0.9 mm, 0.9-1.0 mm, 0.1-0.5 mm, 0.5-1.0 mm, less than 1 mm, more than 1 mm, at least 2 mm, at least 3 mm, at least 4 mm, at least 5 mm, or any range or subrange therebetween of a total catheter distal face 4022. In some embodiments, length 4016 of distal face 4000 is defined by a percentage of the outer diameter of the catheter, for example length 4016 may be equal to a length of 10%-500%, 10%-20%, 20%-30%, 30%-40%, 40%-50%, 50%-60%, 60%-70%, 70%-80%, 80%-90%, 100%-200%, 200%-300%, 300%-400%, 400%-500%, or any range or subrange therebetween of the outer diameter of the catheter.

Referring to FIG. 10B, angle 4020 of the first or angled section of distal face 4000 may be 5-10 degrees, 10-15 degrees, 15-20 degrees, 20-25 degrees, 25-30 degrees, 30-35 degrees, 35-40 degrees, 40-45 degrees, 45-50 degrees, 50-55 degrees, 55-60 degrees, 60-65 degrees, 65-70 degrees, 70-75 degrees, 75-80 degrees, 80-85 degrees, 85-90 degrees, 90-95 degrees, or any range or subrange therebetween. Angle 4020 of the first or angled section of distal face 4000 may be at least 10 degrees, at least 15 degrees, at least 20 degrees, at least 25 degrees, at least 30 degrees, at least 35 degrees, at least 40 degrees, at least 45 degrees, at least 50 degrees, at least 55 degrees, at least 60 degrees, at least 65 degrees, at least 70 degrees, at least 75 degrees, at least 80 degrees, at least 85 degrees, at least 90 degrees, or at least 95 degrees. In some embodiments, angle 4020 of the first or angled section of distal face equals 0-30 degrees, 30-60 degrees, 60-90 degrees, or any range or subrange therebetween. In some embodiments, angle 4020 equals 15-45 degrees, 45-75 degrees, 75-95 degrees, or any range or subrange therebetween. Angle 4010 of the second or blunted section of distal face 4000 may be 55-60 degrees, 60-65 degrees, 65-70 degrees, 70-75 degrees, 75-80 degrees, 80-85 degrees, 85-90 degrees, or any range or subrange therebetween. Angle 4010 of the second or blunted section of distal face 4000 may be at least 55 degrees, at least 60 degrees, at least 65 degrees, at least 70 degrees, at least 75 degrees, at least 80 degrees, at least 85 degrees, or at least 90 degrees. In some embodiments, angle 4010 of the second or blunted section of distal face 4000 equals 55-65 degrees, 65-75 degrees, 75-90 degrees, or any range or subrange therebetween.

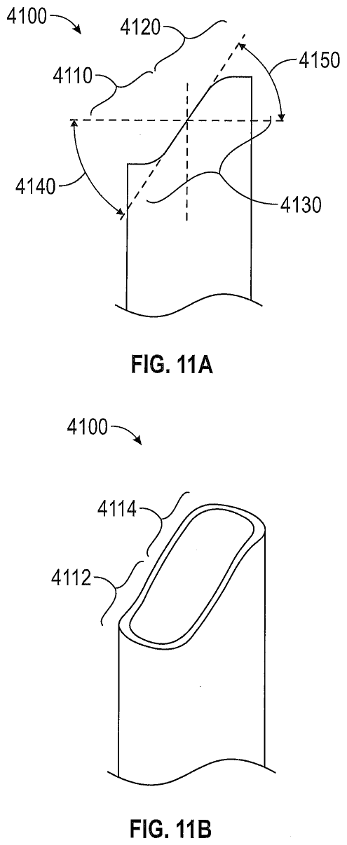

Referring to FIGS. 11A-11B, a sinusoidal distal face 4100 is shown comprising a first section or convex wave or shape or scoop or curve 4110 defined by a first radius of curvature transitioning into a second section or concave wave or shape or scoop or curve 4120 defined by a second radius of curvature. Each curve 4110, 4120 may have a diameter ranging from 0.02 inches to 0.05 inches, for example, depending on catheter size. Each curve 4110, 4120 is further defined by a radius of curvature 4112, 4114, respectively, as shown in FIG. 41B. In some embodiments, a first convex curve 4110 and a second concave curve 4120 have a same or similar or substantially similar (+/−5%) radius of curvature 4112, 4114, respectively. In other embodiments, a first convex curve 4110 and a second concave curve 4120 have a different radius of curvature 4112, 4114, respectively. A 2D profile (e.g., when projected onto the radial plane shown in FIG. 11A) of the distal face shape 4100 is given by the following equation:

$$z = \frac{Δ z}{1 + {e}^{- m \frac{r}{{r}_{c}}}} \qquad ( 1 )$$

where Δz is the overall tip length 4130;

m is a slope factor;

r is a radial position; and

r_{c} is a catheter radius.

By changing the slope factor, a steepness of the wave-shape can be adjusted.

As shown in FIG. 11A, a slope or angle of each curve 4110, 4120 can be defined by angle 4140 and 4150, respectively. For example, for a calculation of each angle 4140, 4150, an axis may be positioned at the transition point, falling along a longitudinal center of the catheter, between a first 4110 and second 4120 curve, such that angle 4140 and 4150 define the curve or shape of the distal face 4100. In some embodiments, angle 4140 is 0-5 degrees, 5-10 degrees, 10-15 degrees, 15-20 degrees, 20-25 degrees, 25-30 degrees, 30-35 degrees, 35-40 degrees, 40-45 degrees, 45-50 degrees, 50-55 degrees, 55-60 degrees, 60-65 degrees, 65-70 degrees, 70-75 degrees, 75-80 degrees, 80-85 degrees, 85-90 degrees, or any range or subrange therebetween. In some embodiments, angle 4140 is 30-60 degrees, 15-85 degrees, or any range or subrange therebetween. In some embodiments, angle 4150 is 0-5 degrees, 5-10 degrees, 10-15 degrees, 15-20 degrees, 20-25 degrees, 25-30 degrees, 30-35 degrees, 35-40 degrees, 40-45 degrees, 45-50 degrees, 50-55 degrees, 55-60 degrees, 60-65 degrees, 65-70 degrees, 70-75 degrees, 75-80 degrees, 80-85 degrees, 85-90 degrees, or any range or subrange therebetween. In some embodiments, angle 4150 is 30-60 degrees, 15-85 degrees, or any range or subrange therebetween.

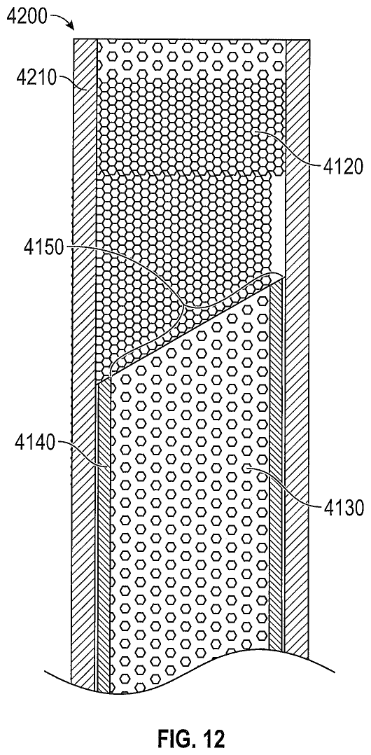

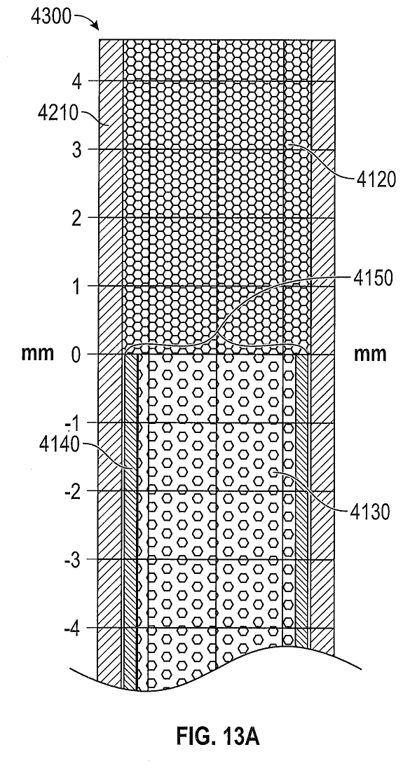

Referring to FIG. 12, a schematic of a numerical simulation model is shown. A Computational Fluid Dynamics (CFD) model was created to simulate the aspiration of a soft clot 4120 in a vessel 4210, surrounded by blood 4130, into a catheter 4140 with various distal face shapes or angles 4150.

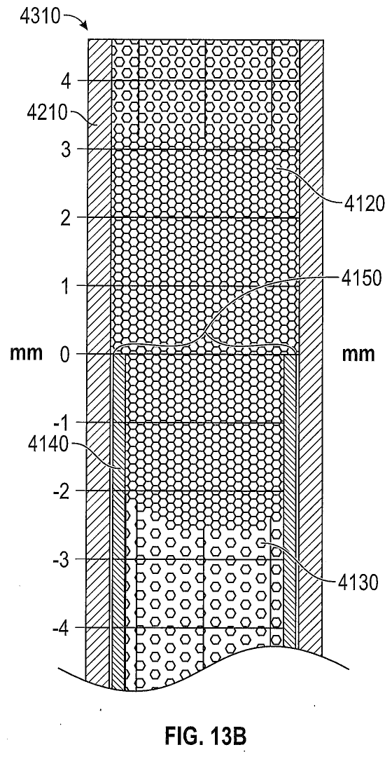

Exemplary CFD model data are shown in FIGS. 13A-13B. Images of Numerical Simulation of clot aspiration show an initial state 4300 in FIG. 13A and an active aspiration state 4310 in FIG. 13B. The amount of clot material aspirated over a given time was measured for various distal face shapes or angles 4150. The value for the circular (transverse) port (e.g., shown in FIG. 3A) was used as a baseline or control to benchmark the success of the proposed profiles. A particular distal face shape or angle was declared more successful if it could aspirate more material then the circular port shape. Greater aspiration rate indicates less resistance to aspiration and a lower chance of complications during an aspiration-thrombectomy.

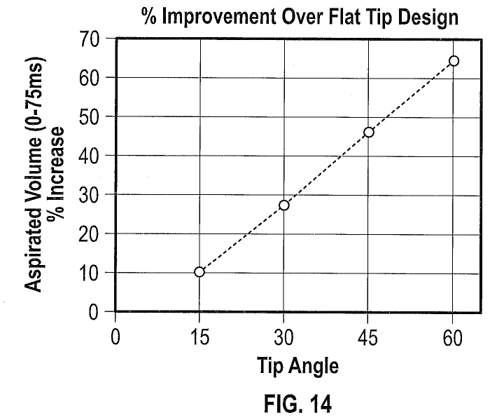

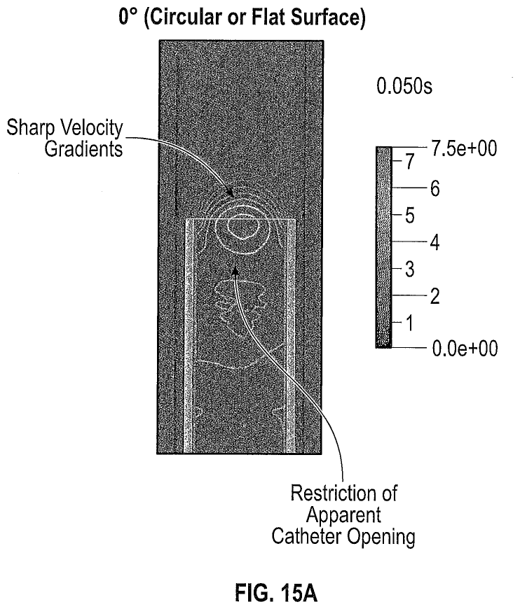

Referring to FIG. 14, the percent increase in aspiration volume is shown as a function of distal face angle. For example, a distal face exhibiting a 60 degree angle had the greatest increase in aspiration efficiency as compared to faces with lesser angles (15 degrees to 45 degrees). The aspirated volume for a 60 degree angled face was increased by greater than 60% over a circular face, between 60-70% over a circular face, or 50-70% over a circular face. A 45 degree angled distal face had an increase in aspirated volume over a circular face of greater than 40%, between 40-50%, between 30-60%, or greater than 45%. A 30 degree angled distal face has an increase in aspirated volume over a circular face of greater than 20%, greater than 25%, between 20-30%, or between 15-40%. Even a distal face having a 15 degree angle showed a 10% improvement in aspirated volume over a circular face.

Exemplary reasoning for this improvement in aspiration efficiency as a function of tip angle is: (1) increases a surface area of the catheter opening in contact with clot which increases the force on the clot (Pressure=Force/Area) and (2) increases a length of distance over which the clot is ingested (i.e., ingestion length) thereby smoothing its change in shape as it flows from the larger diameter vessel into the smaller diameter catheter. The later of these two design-theories is supported by an investigation of the flow profile during the aspiration experiments. For the more pronounced sinusoidal profile, the flow was more uniform and less disrupted upon entering the smaller diameter catheter. As shown in FIG. 14, as face angle increases, the percent increase in aspiration volume increases, collectively indicating that a larger face opening as a result of an angled face profile increases aspiration efficiency.

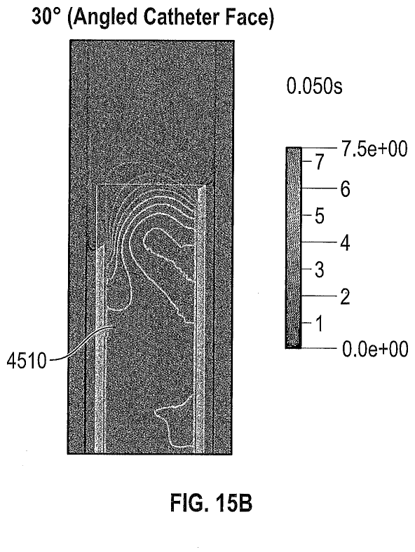

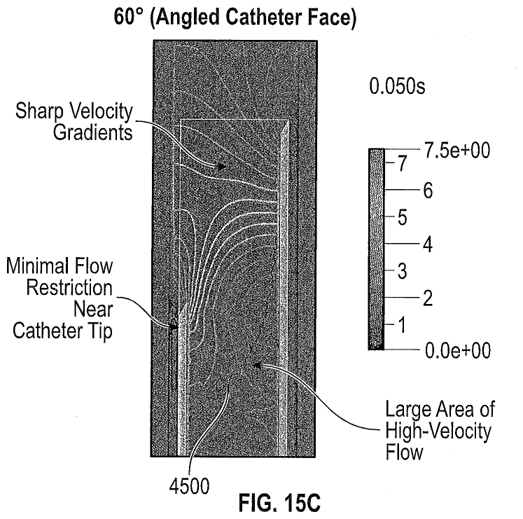

Turning to FIGS. 15A-15C, which show CFD velocity field profile for various face angles (i.e., no angle or 0 degrees, 30 degree angle, and 60 degree angle). As shown in FIG. 15A, the circular face profile showed a significant constriction of the flow thus increasing the resistance to clot ingestion. In contrast, a distal face having an angle of 30 degrees (FIG. 15B) or 60 degrees (FIG. 15C) dramatically increased clot ingestion, as shown in the respective plots. As shown in FIG. 15C, the 60 degree angled face showed greater distance between adjacent contours and a leading edge 4500 of the velocity modulus extended further into the modeled catheter body as compared to the leading edge 4510 of the 30 degree angled face.

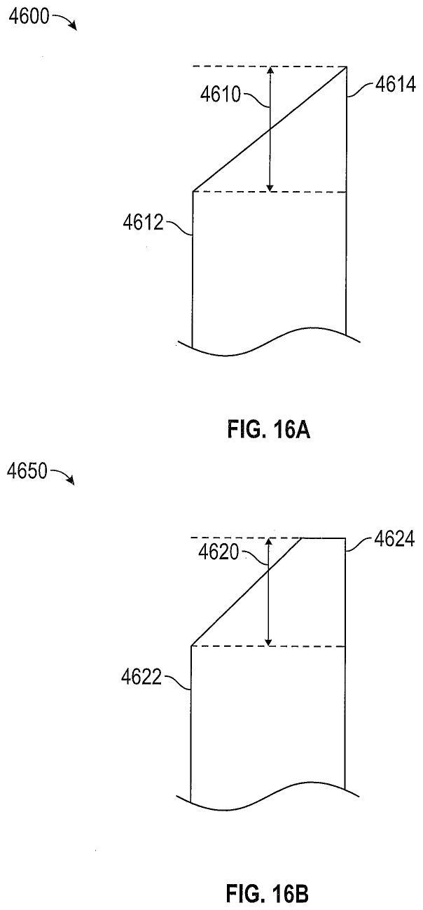

Turning to FIGS. 16A-16B. FIG. 16A shows an ingestion length 4610 for an angled face 4600, and FIG. 16B shows an ingestion length 4620 for a blunted, angled face 4650. In some embodiments, length 4610 is equal to or substantially equal to length 4620; in other embodiments, length 4610 is greater than length 4620, less than length 4620, or different than length 4620. Ingestion length 4610 is determine by calculating a difference between leading edge tip 4614 and trailing edge tip 4612, the leading and trailing edges having been described in connection with FIG. 7E. Similarly, ingestion length 4620 is determined by calculating a difference between leading edge tip 4624 and trailing edge tip 4622. In exemplary, non-limiting embodiments, ingestion length ranges from 0.25 mm to 4.5 mm; 0.5 mm to 4 mm, 0.5 mm to 2.5 mm, 3.5 mm to 4 mm, 2 mm to 2.5 mm, 1.5 mm to 2 mm, 1 mm to 1.5 mm, 0.5 mm to 1 mm, etc. Ingestion length is directly related to percent increase in aspirated volume over circular face control.

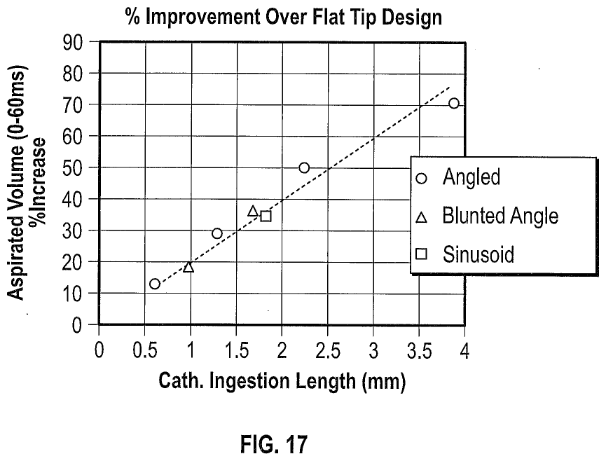

FIG. 17 illustrates the percent increase in aspirated material for catheters with varying distal face profiles (i.e., angled, blunted angled, sinusoidal) as a function of ingestion length. All the proposed profiles showed improvement over the circular face profile. The improvement ranged from substantially 10% to substantially 70%. In addition, the blunted angled face and the sinusoidal face were just as successful as the angled face, which had the same ingestion length. This implies that blunted or smooth catheters can be as successful as sharp catheters as long as the ingestion length is the same, similar, or substantially similar.

For example, as ingestion length for an angled face ranged from about 0.5 mm to about 4 mm and the percent increase in aspirated volume over circular face control increased from about 10% to about 70%. As ingestion length for an angled blunted face ranged from about 1 mm to about 1.6 or about 1 mm to about 1.75 mm and the percent increase in aspirated volume over circular face control increased from about 18% to about 38%. The sinusoidal face demonstrated about a 35% increase in aspirated volume over circular face control for an ingestion length of about 1.7 mm to about 1.7 mm.

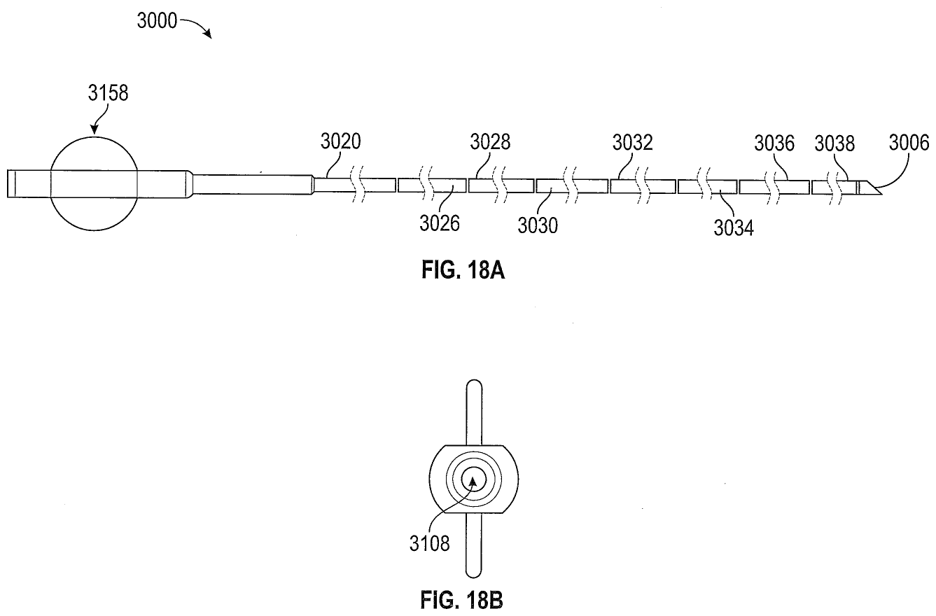

Referring to FIGS. 18A-18B, there is illustrated one example of an outer jacket segment stacking pattern for a progressive flexibility catheter of the type discussed in connection with for example, FIG. 1A or 7A. A distal segment 3038 may have a length within the range of about 1-3 cm, and a durometer of less than about 35D or 30D. An adjacent proximal segment 3036 may have a length within the range of about 4-6 cm, and a durometer of less than about 35D or 30D. An adjacent proximal segment 3034 may have a length within the range of about 4-6 cm, and a durometer of about 35D or less. An adjacent proximal segment 3032 may have a length within the range of about 1-3 cm, and a durometer within the range of from about 35D to about 45D (e.g., 40D). An adjacent proximal segment 3030 may have a length within the range of about 1-3 cm, and a durometer within the range of from about 50D to about 60D (e.g., about 55D). An adjacent proximal segment 3028 may have a length within the range of about 1-3 cm, and a durometer within the range of from about 35D to about 50D to about 60D (e.g., about 55D). An adjacent proximal segment 3026 may have a length within the range of about 1-3 cm, and a durometer of at least about 60D and typically less than about 75D.

More proximal segments may have a durometer of at least about 65D or 70D. The distal most two or three segments may comprise a material such as Tecothane, and more proximal segments may comprise PEBAX or other catheter jacket materials known in the art. At least three or five or seven or nine or more discrete segments may be utilized, having a change in durometer between highest and lowest along the length of the catheter shaft of at least about 10D, preferably at least about 20D and in some implementations at least about 30D or 40D or more.

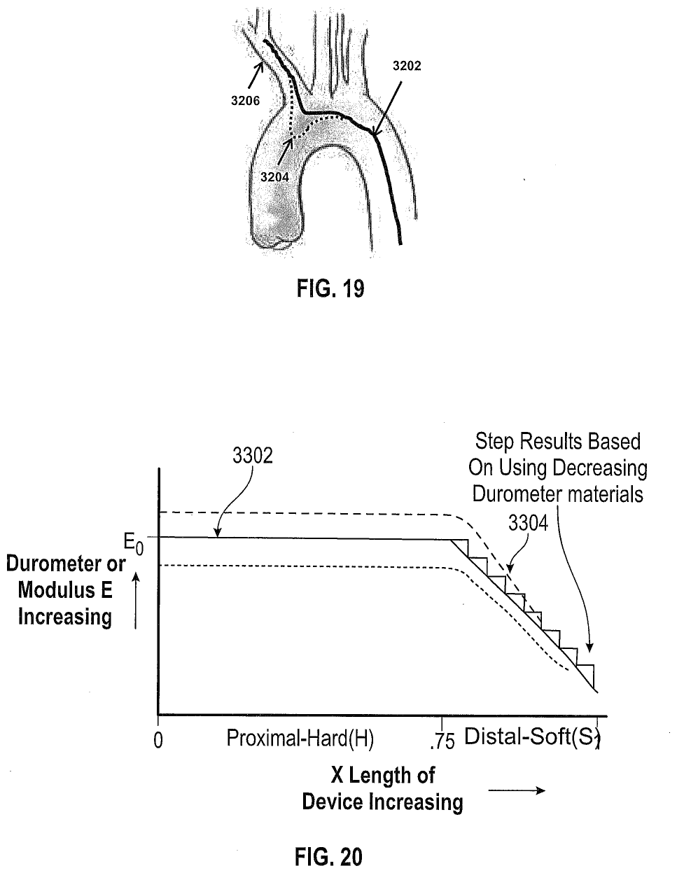

Performance metrics of a catheter include back-up support, trackability, pushability, and kink resistance. Back-up support means ability of the catheter to remain in position within anatomy and provide a stable platform through which endoluminal devices may advance. Referring to FIG. 19, when the devices are pushed through the catheter 3202, if there is not enough back-up support in the catheter 3202, the distal portion 3204 of the catheter 3202 may prolapse, pull out, or back out of a vessel 3206 that branches out of a main blood vessel (e.g., brachiocephalic artery 82, common carotid artery 80, or subclavian artery 84). Back-up support for the catheter 3202 may be improved by providing a proximal region with high durometer or modulus and a distal region with low durometer or modulus.

Durometer or modulus of the proximal region of the catheter 3202 may be improved by braid reinforcement. The region of the catheter at which durometer or modulus is strengthened may be placed near branching points at which the aortic arch 1114, 1214 branches into brachiocephalic artery 82, common carotid artery 80, or subclavian artery 84 or near other anatomical structures (i.e., branching points) at which a main vessel branches into one or more smaller vessels, providing an opportunity for a catheter with poor back-up support to prolapse. For example, the region of the catheter at which durometer or modulus is strengthened may be placed within about 0.5 cm, about 1 cm, about 2 cm, about 3 cm, about 4 cm, about 5 cm, or about 6 cm from a branching point at which a main vessel branches into one or more smaller vessels.

Trackability means ability of the catheter to track further distally than other catheters (e.g., to M1). For example, a catheter that can reach a cerebral segment of the internal carotid artery (ICA) has better trackability than a catheter that can reach a cavernous or petrous segment of the ICA. Trackability of the catheter may be improved by using a catheter wall with low durometer or modulus or by adding a coating (e.g., a hydrophilic coating) on at least a portion of the catheter wall. In one embodiment, the hydrophilic coating may be placed along the distal most region of the catheter. The hydrophilic coating on the catheter may extend to about 1 cm, about 5 cm, about 10 cm, about 15 cm, or about 20 cm from the distal end of the catheter. The region with lower durometer or modulus may locate at the distal most region of the catheter. The region with lower durometer or modulus may extend to about 1 cm, about 5 cm, about 10 cm, about 15 cm, or about 20 cm from the distal end of the catheter.

Pushability means rigidity of the catheter sufficient to push through anatomy without “buckling”. Pushability of the catheter may be improved by increasing its durometer or modulus. Pushability of the catheter may also be improved by providing a proximal region with high durometer or modulus and a distal region with low durometer or modulus. A transition region of the catheter in which durometer or modulus changes along its longitudinal length (e.g., decreasing durometer or modulus from the proximal end to the distal end) may begin at about 50%, 60%, 70%, 75%, 80%, or more of the length of the catheter from its proximal end.

Kink resistance means resistance of the catheter to kinking. In addition, if the catheter does kink, kink resistance of the catheter helps it return to its original shape. Kink resistance is important in the distal segment of the catheter, which is more prone to kinking than the proximal segment. Kink resistance of the catheter may be improved by adding one or more NiTi coils (or a coil at least portion of which is Nitinol) to the catheter wall.

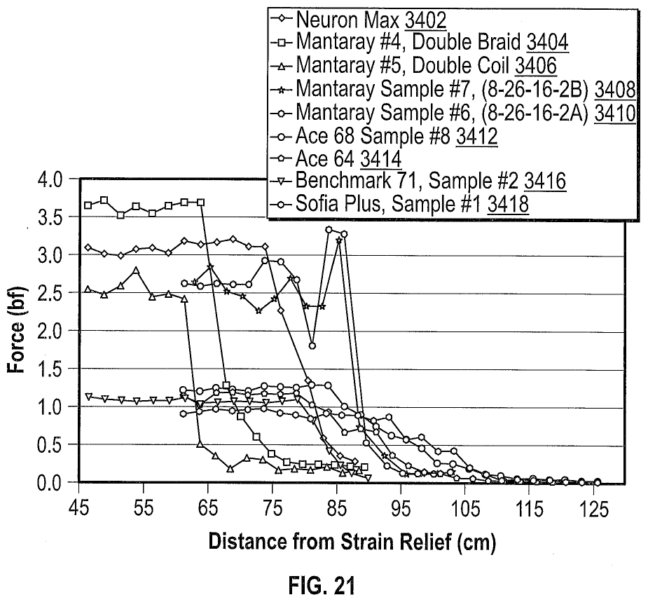

FIG. 20 describes a graph of durometer or modulus of a catheter in accordance with the present invention along the length of the catheter, from the proximal end (x=0) to the distal end (x=1). The catheter according to an embodiment may have a decreasing durometer or modulus (E) approaching its distal end. The proximal end of the catheter has higher durometer or modulus than that of the distal end of the catheter. High durometer or modulus near the proximal end provides superior back-up support of the catheter. Durometer or modulus of the catheter is substantially constant along its length near the proximal end 3302 of the catheter. Then, durometer or modulus of the catheter decreases near the distal end 3304 of the catheter. Durometer or modulus of the catheter may begin to decrease (i.e., transition region) at about 50%, 70%, 75%, 80%, or 90% of the length of the catheter from its proximal end. The catheter may have successively decreasing durometer or modulus near its distal end by using materials with less durometer or modulus or having a thinner catheter wall near the distal end. Decreased durometer or modulus near the distal end provides superior trackability of the catheter.

FIG. 21 describes flexibility test profiles of catheters in accordance with the present invention compared with conventional catheters. Flexibility of a catheter was measured by the three point flexural test with a span of one inch and a displacement of 2 mm. In other words, FIG. 21 describes a force (i.e., flexural load) necessary to vertically displace an one-inch-long catheter segment by 2 mm with respect to distance from strain relief (i.e., proximal end of the catheter) to the point of force application. Modulus of the catheters stays substantially constant along its length near the proximal end and then gradually decreases near the distal end.

Catheters according to the present invention have a flexural load that is substantially constant along the longitudinal length near the proximal end and a rapidly decreasing flexural load near the distal end. In a catheter having a length of about 125 cm, the catheters may have a flexural load greater than or equal to about 1.0 lbF, about 1.5 lbF, about 2.0 lbF, about 2.5 lbF, about 3.0 lbF, or about 3.5 lbF at about 85 cm from the proximal end. The catheters may have a flexural load less than or equal to about 2.5 lbF, about 2.0 lbF, about 1.5 lbF, about 1.0 lbF, or about 0.5 lbF at about 95 cm from the proximal end. The catheters may have a flexural load less than or equal to about 1.5 lbF, about 1.0 lbF, about 0.75 lbF, about 0.5 lbF, about 0.25 lbF, or about 0.1 lbF at about 105 cm from the proximal end. The catheters may have a flexural load less than or equal to about 1.0 lbF, about 0.75 lbF, about 0.5 lbF, about 0.4 lbF, about 0.3 lbF, about 0.2 lbF, or about 0.1 lbF at about 115 cm from the proximal end. For catheters having different lengths, the foregoing dimensions can be scaled from the distal end of the catheter as a percentage of catheter length.

In certain implementations constructed in accordance with FIG. 5, the flexural load is less than about 3.0 or 3.25 lbF at 65 cm from the proximal end and greater than about 2.25 or 2.5 lbF on average from 65 cm to 85 cm from the proximal end. Flexural load drops to no more than about 1.0 and preferably no more than about 0.5 lbF at about 95 cm from the proximal end. This provides enhanced backup support in the aorta while maintaining enhanced trackability into the distal vasculature.

In other embodiments, the catheters may have a flexural load greater than or equal to about 1.0 lbF, about 1.5 lbF, about 2.0 lbF, about 2.5 lbF, about 3.0 lbF, or about 3.5 lbF at about 60 cm from the proximal end. The catheters may have a flexural load less than or equal to about 2.0 lbF, about 1.5 lbF, about 1.0 lbF, or about 0.5 lbF at about 70 cm from the proximal end. The catheters may have a flexural load less than or equal to about 1.0 lbF, about 0.75 lbF, about 0.5 lbF, about 0.4 lbF, about 0.3 lbF, about 0.2 lbF, or about 0.1 lbF at about 80 cm from the proximal end. The catheters may have a flexural load less than or equal to about 1.0 lbF, about 0.75 lbF, about 0.5 lbF, about 0.4 lbF, about 0.3 lbF, about 0.2 lbF, or about 0.1 lbF at about 90 cm from the proximal end.

The catheters may have a transition region, in which its flexural load changes by greater than or equal to about 1.0 lbF, about 1.5 lbF, about 2.0 lbF, about 2.5 lbF, about 3.0 lbF, or about 3.5 lbF. The longitudinal length of the transition region may be less than or equal to about 20 cm, about 15 cm, about 10 cm, about 5 cm, about 3 cm, or about 1 cm.

Compared to Neuron Max (Penumbra, Inc.) 3402, catheters according to the present invention (e.g., 3404, 3406, 3408, 3410) have comparable modulus near its proximal end. This way, the catheters according to the present invention provide back-up support comparable to that of Neuron Max. In addition, the catheters have modulus that falls more rapidly near the transition region (between the proximal end and the distal end) than that of Neuron Max.

Compared to Ace 68 catheter (Penumbra) 3412, Ace 64 catheter (Penumbra) 3414, Benchmark 71 catheter (Penumbra) 3416, and Sofia Plus (MicroVention) 3418, the catheters according to the present inventions have greater modulus near its proximal end and comparable modulus near its distal end. This way, the catheters according to the present invention may provide superior back-up support with comparable trackability compared to conventional catheters. The catheters according to the present invention may achieve this modulus profile even when their inner diameters (and thus lumen volumes) are greater than or equal to those of Ace 68, Ace 64, Benchmark 71, and Sofia Plus, which range from 0.064 inch to 0.071 inch.

Access for the catheter of the present invention can be achieved using conventional techniques through an incision on a peripheral artery, such as right femoral artery, left femoral artery, right radial artery, left radial artery, right brachial artery, left brachial artery, right axillary artery, left axillary artery, right subclavian artery, or left subclavian artery. An incision can also be made on right carotid artery or left carotid artery in emergency situations.

Avoiding a tight fit between the guidewire and inside diameter of guidewire lumen enhances the slidability of the catheter over the guidewire. In ultra small diameter catheter designs, it may be desirable to coat the outside surface of the guidewire and/or the inside surface of the wall defining the GW lumen with a lubricous coating to minimize friction as the catheter 10 is axially moved with respect to the guidewire. A variety of coatings may be utilized, such as Paralene, Teflon, silicone, polyimide-polytetrafluoroethylene composite materials or others known in the art and suitable depending upon the material of the guidewire or inner tubular wall.

Aspiration catheters of the present invention which are adapted for intracranial applications generally have a total length in the range of from 60 cm to 250 cm, usually from about 135 cm to about 175 cm. The length of the proximal segment 33 will typically be from 20 cm to 220 cm, more typically from 100 cm to about 120 cm. The length of the distal segment 34 will typically be in the range from 10 cm to about 60 cm, usually from about 25 cm to about 40 cm.

The catheters of the present invention may comprise any of a variety of biologically compatible polymeric resins having suitable characteristics when formed into the tubular catheter body segments. Exemplary materials include polyvinyl chloride, polyethers, polyamides, polyethylenes, polyurethanes, copolymers thereof, and the like. Optionally, the catheter body may be reinforced with a metal or polymeric braid or other conventional reinforcing layer.

The catheter body may further comprise other components, such as radiopaque fillers; colorants; reinforcing materials; reinforcement layers, such as braids or helical reinforcement elements; or the like. In particular, the proximal body segment may be reinforced in order to enhance its column strength and torqueability (torque transmission) while preferably limiting its wall thickness and outside diameter.

In one aspect of present disclosure, the system for aspirating a vascular occlusion further comprises a controller for applying a pulsatile vacuum cycle to the central lumen. In another aspect of present disclosure, the system for aspirating a vascular occlusion further comprises a rotating hemostasis valve coupled to the proximal end of the tubular body, the rotating hemostasis valve comprising: at least one main lumen along its longitudinal length, through which the proximal portion of the clot grabber is configured to pass, and an aspiration lumen bifurcating from the main lumen and provided with a vacuum port.

In accordance with another aspect, there is provided a method of aspirating material via a femoral access site from at least as distal as the cavernous segment of the internal carotid artery, comprising the steps of: advancing a guidewire from the femoral access site to at least as distal as the cerebral segment of the internal carotid artery, the guidewire having a proximal section having a diameter of at least about 0.030 inches and a distal section having a length of no more than about 25 cm and a diameter of no more than about 0.020 inches; tracking an aspiration catheter directly over the guidewire and to a site at least as distal as the cavernous segment, the aspiration catheter having a distal end and a central lumen at the distal end with a diameter of at least about 0.080 inches and a beveled distal tip. In one aspect of present disclosure, the diameter of the proximal section of the guidewire is about 0.038 inches, and the diameter of the distal section is about 0.016 inches.

With the distal end positioned at least as far distally as the cavernous segment of the middle cerebral artery, vacuum is applied to the lumen to draw thrombus into the lumen; and the thrombus is mechanically engaged to facilitate attachment and potentially entry into the lumen.

The mechanically engaging step may comprise advancing a clot grabber to or beyond the distal end of the tubular body. The method of engaging a vascular occlusion may comprise manually rotating the clot grabber within the tubular body to engage the clot.

In yet another aspect of present disclosure, the method of aspirating a vascular occlusion further comprises providing sufficient back up support to the combined access and aspiration catheter to resist prolapse of the catheter into the aorta. The back up support may be provided to the combined access and aspiration catheter by advancing the combined access and aspiration catheter over a guidewire having a distal end positioned at least as distal as the cavernous segment of the internal carotid artery, and a diameter at the point the guidewire enters the brachiocephalic artery of at least about 0.030 inches. The back up support is provided to the combined access and aspiration catheter by advancing the combined access and aspiration catheter over a guidewire having a distal end positioned at least as distal as the cavernous segment of the internal carotid artery, and a diameter at the point the guidewire enters the brachiocephalic artery of at least about 0.030 inches, such as about 0.035 inches or about 0.038 inches.

The guidewire is navigable to at least the cerebral segment of the internal carotid artery by having a distal segment having a diameter of no more than about 0.020 inches. The guidewire may be navigable to at least the cerebral segment of the internal carotid artery by having a distal segment having a diameter of about 0.016 inches. The diameter of the proximal section of the guidewire may be about 0.038 inches, and the diameter of the distal section may be about 0.016 inches.

Although the present invention has been described in terms of certain preferred embodiments, it may be incorporated into other embodiments by persons of skill in the art in view of the disclosure herein. The scope of the invention is therefore not intended to be limited by the specific embodiments disclosed herein, but is intended to be defined by the full scope of the following claims.

### Example Embodiments

A system for removing embolic material from an intravascular site, comprising one or more of the following:
an elongate, flexible tubular body, having a proximal end, a distal end, and a tubular side wall defining at least one lumen extending axially there through;
an axial restraint carried by the side wall and exposed to the lumen;
a rotatable core wire extendable through the lumen, the core wire having a proximal end and a distal end;
a limit carried by the core wire, the limit having a bearing surface for rotatably engaging the restraint; and
a clot grabbing tip on the distal end of the core wire;
wherein the limit and the restraint are configured to permit rotation of the core wire but limit distal advance of the tip to no more than about 6 mm beyond the distal end of the tubular body.

A system for removing embolic material from an intravascular site as disclosed in any embodiment herein, wherein the limit and the restraint are configured to permit rotation of the core wire but limit distal advance of the tip to no more than about 3 mm beyond the distal end of the tubular body.

A system for removing embolic material from an intravascular site as disclosed in any embodiment herein, wherein the clot grabbing tip comprises a helical thread.

A system for removing embolic material from an intravascular site as disclosed in any embodiment herein, wherein the limit and the restraint are configured to permit rotation of the core wire but limit distal advance of the tip to expose between about one and three full revolutions of the thread beyond the distal end of the tubular body.

A system for removing embolic material from an intravascular site as disclosed in any embodiment herein, wherein the axial restraint comprises a proximally facing bearing surface.

A system for removing embolic material from an intravascular site as disclosed in any embodiment herein, wherein the axial restraint comprises a radially inwardly extending projection.

A system for removing embolic material from an intravascular site as disclosed in any embodiment herein, wherein the axial restraint comprises an annular flange.

A system for removing embolic material from an intravascular site as disclosed in any embodiment herein, wherein the limit comprises a distally facing bearing surface.

A system for removing embolic material from an intravascular site as disclosed in any embodiment herein, wherein the limit comprises a radially outwardly extending projection.

A system for removing embolic material from an intravascular site as disclosed in any embodiment herein, wherein the radially outwardly extending projection is configured for sliding contact with the restraint.

A system for removing embolic material from an intravascular site as disclosed in any embodiment herein, wherein a proximal bearing surface on the axial restraint is within about 30 cm from the distal end of the tubular body.

A system for removing embolic material from an intravascular site as disclosed in any embodiment herein, wherein the proximal bearing surface is within the range of from about 4 cm to about 12 cm from the distal end of the tubular body.

A system for removing embolic material from an intravascular site as disclosed in any embodiment herein, wherein the helical thread has a greatest major diameter that is no more than about 90% of the inside diameter of the lumen, leaving an annular flow path between the tip and the inner surface of the side wall.

A system for removing embolic material from an intravascular site as disclosed in any embodiment herein, wherein the helical thread has a blunt outer edge.

A system for removing embolic material from an intravascular site as disclosed in any embodiment herein, wherein the limit is positioned within about the distal most 25% of the core wire length.

A system for removing embolic material from an intravascular site as disclosed in any embodiment herein, wherein the core wire is removably positionable within the tubular body.

A system for removing embolic material from an intravascular site as disclosed in any embodiment herein, further comprising a handle configured for manual rotation of the core wire.

A system for removing embolic material from an intravascular site as disclosed in any embodiment herein, wherein the helical thread extends through no more than about eight full revolutions.

A system for removing embolic material from an intravascular site as disclosed in any embodiment herein, wherein the helical thread has a major diameter that increases in a proximal direction from a first diameter near the distal tip to a second, greatest major diameter, and then decreases proximally of the greatest major diameter to a third diameter.

A system for removing embolic material from an intravascular site as disclosed in any embodiment herein, wherein an inside diameter of the tubular body adjacent the clot grabbing tip is at least about 0.015″ greater than a maximum outside diameter of the tip.

A torque transmission system for rotationally orienting a distal end of a catheter, comprising one or more of the following:
an elongate, flexible tubular body, having a proximal end, a distal end, and a tubular side wall defining at least one lumen extending axially there through;
a first engagement surface carried by the side wall and exposed to the lumen;
a torque wire extendable through the lumen, the torque wire having a proximal end and a distal end; and
a second engagement surface carried by the torque wire;
wherein distal advance of the torque wire brings the second engagement surface into rotational coupling engagement with the first engagement surface such that rotation of the torque wire in at least a first direction causes rotation of the distal end of the catheter.

A torque transmission system as disclosed in any embodiment herein, wherein the first engagement surface comprises at least one inclined surface.

A torque transmission system as disclosed in any embodiment herein, wherein the first engagement surface is carried by a radially inwardly extending projection.

A torque transmission system as disclosed in any embodiment herein, wherein the projection comprises a ring positioned in the lumen.

A torque transmission system as disclosed in any embodiment herein, wherein the second engagement surface comprises a distally facing surface.

A torque transmission system as disclosed in any embodiment herein, wherein the distally facing surface comprises at least one inclined surface.

A torque transmission system for rotationally orienting a distal end of a catheter, comprising one or more of the following:
an elongate, flexible tubular body, having a proximal end, a distal end, and a tubular side wall defining at least one lumen extending axially there through;
a first connector on the side wall and exposed to the lumen;
a torque wire extendable through the lumen, the torque wire having a proximal end and a distal end; and
a second, complementary connector carried by the torque wire;
wherein coupling the first and second connectors enables rotation of the distal end of the catheter in response to rotation of the torque wire.

A torque transmission system as disclosed in any embodiment herein, wherein the first connector comprises at least one angled tooth.

A torque transmission system as disclosed in any embodiment herein, wherein the first connector comprises a radially inwardly extending projection.

A torque transmission system as disclosed in any embodiment herein, wherein the projection comprises a ring positioned in the lumen.

A torque transmission system as disclosed in any embodiment herein, wherein the ring comprises at least two angled teeth extending in a proximal direction.

A torque transmission system as disclosed in any embodiment herein, wherein the second connector comprises a distally facing surface carried by the torque wire.

A torque transmission system as disclosed in any embodiment herein, wherein the distally facing surface comprises at least one inclined surface.

A torque transmission system as disclosed in any embodiment herein, wherein the second connector is radially outwardly movable.

A torque transmission system as disclosed in any embodiment herein, wherein the second connector comprises an inflatable balloon and the first connector comprises a surface on the side wall.

A torque transmission system as disclosed in any embodiment herein, wherein the first connector comprises the side wall of an axially extending slot configured to receive a projection on the torque wire.

A method of rotationally orienting a catheter, comprising one or more of the following steps:
advancing a catheter to a site in a body lumen, the catheter having a central lumen and a distal end;
advancing a torque wire into the lumen;
engaging a first connector on the torque wire with a second connector on the catheter; and
rotating the torque wire to cause a rotation of the distal end of the catheter.

A system for removing embolic material from an intravascular site, comprising:
an elongate, flexible tubular body, having a proximal end, a distal end, and a tubular side wall defining at least one lumen extending axially there through;
a first engagement surface carried by the side wall and exposed to the lumen;
a tap wire extendable through the lumen, the tap wire having a proximal end and a distal end; and
a second engagement surface carried by the tap wire;
wherein distal advance of the tap wire brings the second engagement surface into contact with the first engagement surface and transfers momentum from the tap wire to the distal end of the tubular body.

A system for removing embolic material from an intravascular site as disclosed in any embodiment herein, wherein the first engagement surface comprises a proximally facing surface.

A system for removing embolic material from an intravascular site as disclosed in any embodiment herein, wherein the first engagement surface is carried by a radially inwardly extending projection.

A system for removing embolic material from an intravascular site as disclosed in any embodiment herein, wherein the first engagement surface comprises an annular flange.

A system for removing embolic material from an intravascular site as disclosed in any embodiment herein, wherein the second engagement surface comprises a distally facing surface.

A system for removing embolic material from an intravascular site as disclosed in any embodiment herein, wherein the distally facing surface is a distal end of the tap wire.

A system for removing embolic material from an intravascular site as disclosed in any embodiment herein, wherein the distally facing surface is on a hammer head carried by the wire.

A system for facilitating distal advance of a catheter, comprising one or more of the following:
an elongate flexible tubular body, having a proximal end, a distal end and a lumen extending therethrough;
a distal restraint within the lumen;
a tap wire, axially movably positionable through the lumen, and having a distal stopper thereon;
wherein distal travel of the distal stopper through the lumen is limited by the distal restraint.

A system for facilitating distal advance of a catheter as disclosed in any embodiment herein, wherein the distal restraint comprises a ring.

A system for facilitating distal advance of a catheter as disclosed in any embodiment herein, wherein the tubular body terminates in an inclined face.

A neurovascular catheter having an atraumatic navigational tip, comprising one or more of the following:
an elongate flexible tubular body, having a proximal end, a distal end and a side wall
defining a central lumen, a distal zone of the tubular body comprising:
a tubular inner liner;
a helical coil surrounding the inner liner and having a distal end,
a tubular jacket surrounding the helical coil, and extending distally beyond the helical coil distal end to terminate in a catheter distal face, and
a tubular radiopaque marker embedded in the tubular jacket in between the distal end of the coil and the distal face,

wherein the catheter distal face comprises a first section that resides on a first plane which crosses a longitudinal axis of the tubular body at a first angle within the range of from about 35 degrees to about 55 degrees, and a second section that resides on a second plane which crosses the longitudinal axis of the tubular body at a second angle within the range from about 55 degrees to about 90 degrees.

A neurovascular catheter having an atraumatic navigational tip as disclosed in any embodiment herein, wherein the marker has a proximal face that is approximately perpendicular to the longitudinal axis and a marker distal face that resides on a plane which crosses the longitudinal axis at an angle within the range of from about 55 degrees to about 65 degrees.

A neurovascular catheter having an atraumatic navigational tip as disclosed in any embodiment herein, wherein the distal face defines a leading edge of the tubular body which extends distally of a trailing edge of the tubular body, the leading edge and trailing edge spaced about 180 degrees apart from each other around the longitudinal axis.

A neurovascular catheter having an atraumatic navigational tip as disclosed in any embodiment herein, wherein an advance segment of the tubular body extends distally beyond the marker band.

A neurovascular catheter having an atraumatic navigational tip as disclosed in any embodiment herein, wherein the advance segment has an axial length within the range of from about 0.1 mm to about 5 mm on the leading edge of the tubular body.

A neurovascular catheter having an atraumatic navigational tip as disclosed in any embodiment herein, wherein the axial length of the advance segment on the leading edge of the tubular body is greater than a length of an advance segment on the trailing edge of the tubular body

A neurovascular catheter having an atraumatic navigational tip as disclosed in any embodiment herein, wherein an axial length of the marker band on the leading edge of the tubular body is at least about 20% longer than the axial length of the marker band on the trailing edge of the tubular body.

A neurovascular catheter having an atraumatic navigational tip as disclosed in any embodiment herein, wherein the axial length of the marker band on the leading edge of the tubular body is within the range of from about 1 mm to about 5 mm.

A neurovascular catheter having an atraumatic navigational tip as disclosed in any embodiment herein, wherein the marker band comprises at least one axial slit.

A neurovascular catheter having an atraumatic navigational tip as disclosed in any embodiment herein, wherein the tubular liner is formed by dip coating a removable mandrel.

A neurovascular catheter having an atraumatic navigational tip as disclosed in any embodiment herein, wherein the tubular liner comprises PTFE.

A neurovascular catheter having an atraumatic navigational tip as disclosed in any embodiment herein, further comprising a tie layer between the inner liner and the helical coil.

A neurovascular catheter having an atraumatic navigational tip as disclosed in any embodiment herein, wherein the tie layer has a wall thickness of no more than about 0.005 inches.

A neurovascular catheter having an atraumatic navigational tip as disclosed in any embodiment herein, wherein the tie layer extends along at least the most distal 20 cm of the flexible body.

A neurovascular catheter having an atraumatic navigational tip as disclosed in any embodiment herein, wherein the coil comprises Nitinol.

A neurovascular catheter having an atraumatic navigational tip as disclosed in any embodiment herein, wherein the Nitinol comprises an Austenite state at body temperature.

A neurovascular catheter having an atraumatic navigational tip as disclosed in any embodiment herein, wherein the outer jacket is formed from at least five discrete axially adjacent tubular segments.

A neurovascular catheter having an atraumatic navigational tip as disclosed in any embodiment herein, wherein the outer jacket is formed from at least nine discrete axially adjacent tubular segments.

A neurovascular catheter having an atraumatic navigational tip as disclosed in any embodiment herein, wherein the difference in durometer between a proximal one of the tubular segments and a distal one of the tubular segments is at least about 20D.

A neurovascular catheter having an atraumatic navigational tip as disclosed in any embodiment herein, wherein the difference in durometer between a proximal one of the tubular segments and a distal one of the tubular segments is at least about 30D.

A neurovascular catheter having an atraumatic navigational tip as disclosed in any embodiment herein, further comprising a tension support for increasing the tension resistance in the distal zone.

A neurovascular catheter having an atraumatic navigational tip as disclosed in any embodiment herein, wherein the tension support comprises an axially extending filament.

A neurovascular catheter having an atraumatic navigational tip as disclosed in any embodiment herein, wherein the axially extending filament is carried between the inner liner and the helical coil.

A neurovascular catheter having an atraumatic navigational tip as disclosed in any embodiment herein, wherein the axially extending filament increases the tensile strength of the tubular body to at least about 2 pounds.

What is claimed is:

1. A system for removing embolic material from an intravascular site, comprising:
an elongate, flexible tubular body, having a proximal end, a distal end, and a tubular side wall defining at least one lumen extending axially there through;
an axial restraint carried by the side wall and exposed to the lumen;
a rotatable core wire extendable through the lumen, the core wire having a proximal end and a distal end;
a limit carried by the core wire, the limit having a bearing surface for rotatably engaging the restraint; and
a clot grabbing tip on the distal end of the core wire;
wherein the limit and the restraint are configured to permit rotation of the core wire but limit distal advance of the tip to no more than about 6 mm beyond the distal end of the tubular body.

2. A system as in claim 1, wherein the limit and the restraint are configured to permit rotation of the core wire but limit distal advance of the tip to no more than about 3 mm beyond the distal end of the tubular body.

3. A system as in claim 1, wherein the clot grabbing tip comprises a helical thread.

4. A system as in claim 3, wherein the limit and the restraint are configured to permit rotation of the core wire but limit distal advance of the tip to expose between about one and three full revolutions of the thread beyond the distal end of the tubular body.

5. A system as in claim 1, wherein the axial restraint comprises a proximally facing bearing surface.

6. A system as in claim 5, wherein the axial restraint comprises a radially inwardly extending projection.

7. A system as in claim 6, wherein the axial restraint comprises an annular flange.

8. A system as in claim 1, wherein the limit comprises a distally facing bearing surface.

9. A system as in claim 8, wherein the limit comprises a radially outwardly extending projection.

10. A system as in claim 9, wherein the radially outwardly extending projection is configured for sliding contact with the restraint.

11. A system as in claim 1, wherein a proximal bearing surface on the axial restraint is within about 30 cm from the distal end of the tubular body.

12. A system as in claim 11, wherein the proximal bearing surface is within the range of from about 4 cm to about 12 cm from the distal end of the tubular body.

13. A system as in claim 3, wherein the helical thread has a greatest major diameter that is no more than about 90% of the inside diameter of the lumen, leaving an annular flow path between the tip and the inner surface of the side wall.

14. A system as in claim 3, wherein the helical thread has a blunt outer edge.

15. A system as in claim 1, wherein the limit is positioned within about the distal most 25% of the core wire length.

16. A system as in claim 1, wherein the core wire is removably positionable within the tubular body.

17. A system as in claim 1, further comprising a handle configured for manual rotation of the core wire.

18. A system as in claim 3, wherein the helical thread extends through no more than about eight full revolutions.

19. A system as in claim 3, wherein the helical thread has a major diameter that increases in a proximal direction from a first diameter near the distal tip to a second, greatest major diameter, and then decreases proximally of the greatest major diameter to a third diameter.

20. A system as in claim 19, wherein an inside diameter of the tubular body adjacent the clot grabbing tip is at least about 0.015″ greater than a maximum outside diameter of the tip.
